(19) United States
(12) Patent Application Publication
(10) Pub. No.: US 2023/0400453 A1
(43) Pub. Date: Dec. 14, 2023

(54) Method and Kit for Testing Immunomodulatory Potency of Immunoglobulin Compositions for Treatment of COVID-19

(71) Applicant: Biotest AG, Dreieich (DE)

(72) Inventors: Fabian BOHLÄNDER, Erbach (DE); Stefanie FAUST, Dieburg (DE)

(21) Appl. No.: 18/249,806

(22) PCT Filed: Nov. 18, 2021
(86) PCT No.: PCT/EP2021/082154

§ 371 Date: Apr. 20, 2023

(30) Foreign Application Priority Data
Nov. 19, 2020 (EP) 20208703.7

(51) Int. Cl. G01N 33/543; G01N 33/96

(52) U.S. Cl. CPC G01N 33/54313; G01N 33/96; G01N 2333/165; C07K 2317/21

(57) ABSTRACT

The present invention relates to the field of immunotherapeutics, in particular to a method for characterization and/or quality control of immunotherapeutics. It provides a method of testing potency of an immunoglobulin composition, e.g., plasma or a plasma-derived immunoglobulin composition such as an intravenous immunoglobulin composition (IVIG), as well as to use of a bead coated with an antigen and an antibody specifically bound to said antigen for testing po-tency of an immunoglobulin composition. Said immunoglobulin composition, or immunoglobulin test composition can be an IVIG, particularly and IgA- and/or IgM enriched (also sometimes re-ferred to as IVIG-AM). The potency is tested by the capability of the composition to inhibit an ef-fector function of an Fc-receptor expressing immune effector cell, such as a neutrophil, e.g., a HL60 cell, preferably, production of an inflammatory cytokine such as IL-8. The invention also relates to a method of preparing a standardized immunoglobulin composition, to a kit for carry-ing out the method, as well as a composition. The immunoglobulin compositions obtainable from said method may be used, e.g., in the treatment of inflammation, e.g., in the context of COVID-19 or pneumonia, e.g., severe community-acquired pneumonia.

The present invention relates to the field of immunotherapeutics, in particular to a method for characterization and/or quality control of immunotherapeutics. It provides a method of testing potency of an immunoglobulin composition, e.g., plasma or a plasma-derived immunoglobulin composition such as an intravenous immunoglobulin composition (IVIG), as well as to use of a bead coated with an antigen and an antibody specifically bound to said antigen for testing potency of an immunoglobulin composition. The immunoglobulin composition to be tested, or immunoglobulin test composition, can be an IVIG, particularly and IgA- and/or IgM enriched (also sometimes referred to as IVIG-AM). The potency is tested by the capability of the composition to inhibit an effector function of an Fc-receptor expressing immune effector cell, such as a neutrophil, e.g., a HL60 cell, preferably, production of an inflammatory cytokine such as IL-8. The invention also relates to a method of preparing a standardized immunoglobulin composition, to a kit for carrying out the method, as well as a composition. The immunoglobulin compositions obtainable from said method may be used, e.g., in the treatment of inflammation, e.g., in the context of COVID-19 or pneumonia, e.g., severe community-acquired pneumonia.

Antibodies (synonymously: immunoglobulins) have been used for the treatment of many disorders, in particular immunological disorders, but also in the treatment of infectious diseases. The skilled person generally distinguishes monoclonal antibodies (such as derived from a single cell clone or by recombinant means) and polyclonal antibodies (such as typically derived from blood plasma samples). Polyclonal antibodies may be administered in the form of human or animal plasma or serum. More typically, however, they are administered as purified immunoglobulin compositions for intravenous, subcutaneous or intramuscular use, from healthy, infected or immunized donors. Purified plasma-derived immunoglobulin compositions comprising IgG, and optionally also IgA and IgM (often generally summarized under the term IVIG), have been shown to be beneficial in the treatment of certain immunological disorders and neurological disorders (such as Idiopathic Thrombocytopenic Purpura (ITP), Chronic inflammatory demyelinating polyneuropathy (CIDP) and Multifocal motor neuropathy (MNN)), but also in the treatment of infectious diseases, in particular viral diseases. Also known are hyperimmune globulins with defined titers against certain pathogens such as Cytomegaly Virus (CMV), Hepatitis B Virus (HBV) oder Varizella Zoster Virus (VZV). Regarding viral disorders, the recent COVID-19 pandemic has caused activities to develop a hyperimmune globulin against the responsible pathogen, SARS-CoV-2. With respect to bacterial pathogens, an IVIG composition comprising IgM and IgA (also termed IVIG-AM), Pentaglobin® (Biotest AG, Dreieich, Germany), is approved for treatment of bacterial infections and is usually administered as adjunctive therapy to antibiotic treatment.

It is hypothesized that plasma-derived polyclonal immunoglobulin compositions exert their effects through several different modes of action, e.g. through direct binding and neutralization of pathogens, e.g. opsonization, through dampening an overstimulated immune response (immunomodulation), e.g. by scavenging of complement factors or pro-inflammatory cytokines, or through neutralization of viral or bacterial debris or toxins (such as endo- or exotoxins, e.g. lipopolysaccharide or pneumolysin.

Suitable immunoglobulin compositions are typically derived from the plasma or serum of a plurality of donors. Most commercial immunoglobulin compositions contain purified IgG, but there are also compositions containing IgM and IgA, such as Pentaglobin (Biotest AG, Dreieich, DE). Pentaglobin is a composition treated with beta-propiolactone. A different composition comprising IgG, IgA, and IgM is in development (trimodulin, Biotest AG, Dreieich, DE) and has recently entered a clinical study in COVID-19 patients (ESsCOVID-study) (Press Release Biotest).

Suitable antibodies may be administered in the form of human or animal plasma or serum. More typically, however, they are administered as purified immunoglobulin compositions for intravenous, subcutaneous or intramuscular use, for example as human IVIG (intravenous immunoglobulin) from healthy, infected or immunized donors. Most available compositions contain purified IgG, but there are also immunoglobulin compositions comprising not only IgG, but also IgM and IgA (e.g. WO 2011/131786 A2 and WO 2011/131787 A2).

Polyclonal immunoglobulins derived from animal plasma can lead to immune responses in a human subject, and their use in human therapy is, therefore, limited, e.g. to horse-derived anti-venoms.

Immunoglobulin compositions been found to be beneficial in the field of intensive care medicine. E.g., in a recent phase II trial, the adjunctive administration of a novel immunoglobulin composition comprising IgM, IgA, and IgG has been implicated with a reduced mortality in certain patient groups suffering from severe pneumonia, in particular severe community acquired pneumonia (sCAP), (Welte et al., 2018. Intensive Care Med. 44(4):438-448, WO 2017/157850 A1).

With the new SARS-CoV-2 virus induced COVID-19 pandemic, another disease came into the focus of IVIG and IVIG-AM therapy. Severe COVID-19 patients suffer from an excessive immune response which is characterized by hyperinflammation and cytokine storm (McGonagle et al. 2020. Autoimmun. Rev. 19, 102537). It is presently being tested for treatment of pneumonia or acute respiratory distress syndrome (ARDS) in the ESsCOVID (Escape from severe COVID-19) trial for treatment of COVID-19 patients.

Here, the focus is on the immunomodulatory mode of action. The rapid global spread of SARS-CoV-2 virus induced COVID-19 diseases lead to an urgent need for appropriate therapeutics that are available fast. Anti-viral activity of IVIG against common viruses is reported (Gelfand 2012. N. Engl. J. Med. 367, 2015-2025;). But so far, there are no specific antibodies against SARS-CoV-2 in immunoglobulin preparations produced with plasma collected before pandemic. Therefore the usage of IVIG or IVIG-AM focuses on treatment of severe COVID-19 induced symptoms (Lin et al. 2020. Emerg. Microbes Infect. 9, 727-732). A major issue for severe COVID-19 patients is excessive inflammation associated with a cytokine storm, ARDS and, ultimately, respiratory failure (McGonagle et al. 2020. Autoimmun. Rev. 19, 102537). The anti-inflammatory abilities of IVIG or IVIG-AM preparations have been used for treatment of inflammatory diseases for decades; Gelfand 2012. N. Engl. J. Med. 367, 2015-2025.; Schwab and Nimmerjahn 2013. Nat. Rev. Immunol. 13, 176-189). Clinical efficiency and in vitro data from Duerr and colleagues show that trimodulin is a promising immunomodulatory drug in bacterial induced inflammation (Duerr et al 2019. Innate Immun. 25, 374-388.; Welte et al. 2018. Intensive Care Med. 44, 438-448)). Rieben et al., 1999 (Blood 93(3):942-951) showed that IVIG-AM preparations are more efficient than IVIG in preventing complement activation. In contrast, the knowledge about immunomodulation in inflammation induced by viruses is very limited. Immunomodulatory activity of Pentaglobin® in SARS-CoV-virus induced inflammatory disease was reported (Ho et al. 2005. INT J TUBERC LUNG DIS 8(10):1173-1179). Furthermore, first clinical studies show promising results in treatment of COVID-19 patients with IVIG preparations (Cao et al. 2020. Open Forum Infect. Dis. 7.; Xie et al. 2020. J. Infect. S0163-4453(20)30172-9).

The manufacturing of plasma-derived immunoglobulins is generally known. Most commercial methods employ a purification scheme based on Cohn fractionation, usually involving further steps such as chromatography, low pH treatment, octanoic acid treatment, nanofiltration, affinity chromatography and more. Such steps are necessary to remove coagulation factors, blood group specific antibodies and to ensure virus depletion and viral safety. Modern commercial production processes are highly controlled and standardized, ensuring a high degree of safety and constant efficacy over different production batches. Although one may come to the conclusion that today's automated and well-controlled production processes, from plasma donation to final product, would suffice to ensure highest quality, it is important to employ further levels of quality control. This does not only involve structural analyses (such as fragmentation, oxidation and aggregation): In view of the broad spectrum of different activities conveyed by plasma-derived immunoglobulins, and in view of the product being derived from a natural source (blood, blood serum, or blood plasma) it is important to monitor and ensure functional integrity and standardisation of the product throughout its production process up to the final product. During development of an immunoglobulin composition, is also important to establish a method for determining biological activities of the product, and thus to provide a functional product profile.

IgG, IgA, and particularly IgM, are large molecules perceptible to heat, shear stress, enzymatic degradation and other influences during the entire production cascade. An important part of the function of immunoglobulins is mediated by specific binding with target antigens and by effector functions involving binding to receptors and complement factors. Despite stringent controls in manufacturing and even in the absence of changes to the molecule visible in standard analytics, there is a need to ensure that no conformational changes or other modifications have occurred that would affect the biological activity of the immunoglobulin. Further, donor pools differ, e.g., with regard to their prior exposure to a specific antigen, and in consequence, with regard to the content of antibodies directed to said antigen, both qualitatively and quantitatively.

Whereas assays directed at the structural analysis of biological products are commonly known and often well established, it remains a challenge to develop methods to determine and quantitate biological functions of a certain product. Therefore, there is an unmet need to provide functional assays allowing to characterize and monitor certain biological activities, in particular activities which are considered to reflect a pharmaceutically relevant property of the active ingredient. This task is further complicated by the need that such assay should be simple, reliable, reproducible, sufficiently sensitive and should not involve the use of animal testing.

WO 2011/131787 A2 determines the presence and absence of antibodies to specific bacterial antigens by ELISA. However, it is silent about assays testing the functional potency of the antibody product. Grace et al., 2019 (J Vector Borne Dis 56:105-110) and Liu et al., 2015 (Metabolomics 5:2-9) teach use of a latex agglutination test for assessing the presence of neutralizing antibodies against an antigen, e.g., for diagnostic purposes. Nie et al. 2020 (Emerging Microbes & Infections 9:680-686) use a pseudovirus system for evaluating neutralizing antibodies against SARS-CoV-2. However, these assay does not assess immunomodulatory characteristics of antibody compositions.

A potency assay involving the ability of a plasma-derived immunoglobulin composition to neutralize pneumolysin is described in PCT/EP2020/065568 (yet unpublished).

However, there is no satisfactory functional assay to test the immune-modulatory aspect of the mode of action of immunoglobulin compositions. Furthermore, there is a particular need to study the functional integrity of the IgM component in compositions comprising IgM, because IgM is particularly vulnerable (e.g. to heat or shear stress), and it is also considered a highly potent component in an immunoglobulin composition. Furthermore, there is also a particular need to study the functional integrity of the IgA component in compositions comprising IgA, because IgA is an important component in context of immunomodulation. In contrast to pure IgG compositions (such as typical IVIGs), there is much less experience with therapeutic compositions comprising IgM and/or IgA, in particular chemically unmodified compositions comprising IgM and IgA (Pentaglobin® is chemically modified using beta-propiolactone),

The skilled person is thus faced with the problem of monitoring and improved monitoring of the functional integrity and activity of plasma-derived immunoglobulin compositions, in particular immunoglobulin compositions derived from a plurality of donors, in particular immunoglobulin compositions comprising IgM and/or IgA, e.g., if used in medicine.

This problem is solved by the present invention, in particular, by the subject matter of the claims. The present invention provides, in a first embodiment, a method for testing potency of an immunoglobulin test composition, the method comprising
a) providing a bead coated with an antigen and an antibody specifically bound to said antigen,
b) contacting said bead with said immunoglobulin test composition and with an immune effector cell expressing at least one Fc-Receptor (FcR), and
c) determining an effector function of the immune effector cell.

The method of the invention may be advantageously used for characterisation and/or quality control of immunoglobulin compositions, more specifically of polyclonal immunoglobulin compositions, even more specifically of plasma derived-immunoglobulin compositions. Such immunoglobulin composition to be tested are in the following referred to as “immunoglobulin test compositions” in order to distinguish the immunoglobulin composition to be tested from the antibody bound to the antigen coated on the bead provided in step a), because this antibody may be provided in the form of an antibody composition (which technically speaking would also be an immunoglobulin composition), as will be further described below.

To make it more clear, it is important to avoid confusion between antibodies that bind to the antigen coated on the bead (thus forming an immune complex) and the immunoglobulin composition that is to be tested (i.e., the immunoglobulin test composition), as these are used in different steps and have different functions in the assay. Accordingly, we will generally refer to the immunoglobulin composition to be tested as an “immunoglobulin test composition” comprising “immunoglobulins”, whereas we will generally refer to antibody specifically binding to the antigen coated on the bead (e.g. in step (a) of the method above) as an “antibody” or “antibody composition”.

In the context of the invention, the immunoglobulin test composition preferably is a plasma-derived polyclonal immunoglobulin composition. More particularly, the immunoglobulin test composition may comprise IgG and/or IgM and/or IgA, preferably IgG, IgA, and IgM, as further detailed below.

For example, the method according to the invention is very useful for quality control in production of plasma-derived immunoglobulin compositions, e.g., an IVIG or IVIG-AM compositions. Such compositions are known in the art and used in medicine, e.g., as described above. E.g. a representative sample of a batch of immunoglobulin composition manufactured may be provided as an immunoglobulin test composition and subjected to the inventive method in order to thus characterize and/or control the quality of the batch manufactured. Thus, the method can be used to determine the properties and quality, in particular, the potency of immunoglobulin test compositions, i.e., for testing if an immunoglobulin composition derived from a plurality of human donors complies with pre-defined standards, e.g., if it has a desired potency or activity. More particularly, the immune modulatory potency of the composition can be determined. Advantageously, the method of the invention has been found to be particularly sensitive for the potency of the IgM and IgA component in an IVIG-AM composition. The potency can also be seen as a measure for functional integrity, which can accordingly also be tested with the method of the invention.

The methods of the invention may also be used for characterisation, in particular functional characterization, when it is of interest to determine the functional properties of an immunoglobulin test composition, e.g., for providing a product profile of a product not yet functionally characterised.

More specifically, the assay of the invention thus analyses the potency of an immunoglobulin test composition to modulate the immune system by working with a model system, e.g., based on neutrophil-like HL60 cells as immune effector cells. To show that the immunomodulatory effects are applicable to virus-induced inflammation, a virus-like model, e.g., a SARS-CoV-2 virus-like model is provided. Antigen, such as recombinant SARS-CoV-2 spike protein, is coated on beads that mimic the virus particle, e.g., fluorescent latex beads. The immunoglobulin test composition efficiently inhibits inflammation reducing effector functions of the immune effector cell, such as cytokine secretion.

The inventors have found this method to be simple, reliable, reproducible, and sensitive. The method does not involve the use of animal testing. Most components can be obtained in standardized quality and can be controlled for their quality by known methods. The only living component, the immune effector cell, can be provided using well-established immortal cell lines, allowing for a good degree of standardization and reproducibility.

It is of particular significance that, with the method of the invention, the comparison of immunomodulatory potency between compositions comprising only IgG, such as standard IVIG, and compositions comprising, in addition to IgG, significant amounts of IgM and IgA, can show clear benefits of non IgG-classes of immunoglobulin for the potency of the composition. Without intending to be bound by the theory, the multimeric immunoglobulins in IgM- or IgA-comprising immunoglobulin test compositions may have a better ability to displace immune complex from Fc-receptors, hence more monomeric IgG and IgA species remain to target free Fc-receptors, which may result in inhibitory immunoreceptor tyrosine-based activation motif (ITAMi) signalling. In addition to common FcγRs, IgA is able to target the FcαRI, which has multiple important functions in regulating immunity (Breedveld and van Egmond 2019. Front. Immunol. 10, 553). Especially in regulating respiratory diseases, IgA seem to be a key player.

The invention also provides use of a bead coated with an antigen and an antibody specifically bound to said antigen for quality control and of characterization of an immunoglobulin test composition-. Preferably, in the context of quality control, it is used for testing potency of an immunoglobulin test composition, in particular, immunomodulatory potency. Said use may comprise the steps of the method of the invention, e.g.,
a) providing a bead coated with an antigen and an antibody specifically bound to said antigen,
b) contacting said bead with said immunoglobulin test composition and with an immune effector cell expressing at least one FcR, and
c) determining an effector function of the immune effector cell, as further detailed herein.

The potency may then be determined based on the effector function of the immune cell.

The bead coated with an antigen and an antibody specifically bound to said antigen

In the context of the invention, the antigen used for coating the bead may be chosen as deemed appropriate by the skilled person, e.g. the antigen may be chosen to reflect a particular therapeutic situation. Preferably the antigen is derived from a pathogen, e.g., any bacterial antigen or any viral antigen. It may be a virus surface protein, such as spike glycoprotein, a nucleocapsid protein, membrane protein or an envelope protein. The virus may be a coronavirus, e.g., SARS-CoV-2. Alternatively, the antigen may be a bacterial antigen, e.g. endotoxin (lipopolysaccharides), cell wall proteins or other pathogen associated molecular patterns (PAMPs). The antigen may also be an antigen targeted by an autoimmune response.

In one embodiment of the invention, the antigen is a SARS-CoV-2 surface protein, such as SARS-CoV-2 spike protein. Said protein is the main target of antibody responses in COVID-19 (Saghazadeh, A., and Rezaei, N. (2020) International Immunopharmacology 84, 106560). It may thus play an important part in the pathogenesis of overwhelming immune responses that may be induced by SARS-CoV-2. For example, the antigen may be full length SARS-CoV-2 spike protein, optionally further including a purification tag, such as a His-tag. It may alternatively only comprise the receptor-binding domain (RBD) of SARS-CoV-2 spike protein. Typically, the antigen is a recombinant antigen, i.e. a protein or glycoprotein moiety manufactured by recombinant methods.

The antigen may also be a different virus antigen, e.g., from influenza (e.g., a HA protein or neuraminidase), hepatitis B or C virus, CMV, EBV, MERS or SARS-CoV-1. Infection with certain viruses, including SARS-CoV-2, SARS-CoV-1, MERS or hepatitis B/C virus, may lead to inflammation including bystander activation and an strong and damaging, potentially overwhelming immune response. It may be advantageous to use antigens from such viruses in the assay of the invention. However, without intending to be bound by theory, the immune stimulation of the effector cell in the present method is thought to be largely mediated by the Fc-parts of the antibodies in the immune complex formed on the bead. Thus, the immune modulating potency of the immune globulin test composition analyzed in the present method is largely independent of the specific type of antigen on the bead.

According to the invention, the bead is coated with the antigen and an antibody specifically bound to said antigen, i.e., an antibody which is capable of specifically binding to the antigen. The antibody and the antigen thus form an immune complex. In this immune complex, the Fc part of the antibodies is not bound, i.e., it is free to bind to other binding partners like Fc-receptors.

The antibody used in this context should be capable of binding to a human Fc receptor. Typically, it is a human or humanized antibody.

In the context of the invention, “a” or “one” is generally understood to refer to “at least one”, if not explicitly mentioned otherwise. Thus, the antibody may be monoclonal or polyclonal (i.e. a plurality of different antibodies with different amino acid sequence). The antibody may be purified or partly purified, e.g. the antibody may be comprised in heat inactivated plasma added to the antigen-coated bead.

The antibody specifically bound to said antigen coated on the bead may be an antibody from a patient who has been infected with a pathogen expressing said antigen. For example, if the antigen is a SARS-CoV-2 surface protein, such as SARS-CoV-2 spike protein, it may be antibody derived from a subject who has been infected with SARS-CoV-2, and who has produced antibodies to said surface protein. For example, in the case of SARS-CoV-2, the antibody can be comprised in heat-inactivated plasma of a convalescent COVID-19 patient, or derived therefrom. Heat-inactivation may, e.g., be carried out for about 30 min at 56° C. Heat-inactivation inactivates complement components that are otherwise contained in human plasma. The antibody may also be comprised in an antibody composition purified from human blood plasma.

Specifically bound means binding via the antigen binding sites with a sufficient affinity that allows for binding of the antibodies to the antigen without significant dissociation of the immune complex during the method of the invention, so as not to impair the read-out of the method. For example, specific binding may be binding with an affinity of at least 10⁻⁶ M, e.g., 10⁻⁷ M, at least 10⁻⁸ M, at least 10⁻⁹ M or at least 10⁻¹⁰ M.

The antibody can be of one or more classes, such as IgG, IgM and/or IgA, in particular it may also be a mixture of classes, e.g., as contained in plasma. As this mirrors the situation in a virus infection, it may provide particularly relevant results. For example, if potency for therapy of a certain pathogen is of interest, plasma (or an immunoglobulin composition derived from it) from a subject having specific antibodies against such pathogen, e.g., from a vaccinated subject or a convalescent patient may be used, preferably comprising IgG, IgA, and IgM, because all three classes are relevant for neutralizing the pathogen. For example, if potency for therapy of SARS-CoV-2 is of interest, plasma from a vaccinated or a convalescent SARS-CoV-2 infected subject, e.g., a former COVID-19 patient, or Ig of all classes derived from it, are preferably used, because all three classes (IgG, IgA, IgM) are relevant for neutralizing the pathogen. Therefore, the immunomodulatory effect against all antibody classes can be advantageously assessed. This may be of particular interest if potency is analyzed in the context of characterizing an immunoglobulin test composition.

On the other hand, use of monoclonal or recombinant antibodies may allow for easier standardisation of the assay. Thus, the antibody specifically bound to said antigen may also be a monoclonal or recombinant antibody. Optionally, it is a mixture of different recombinant antibodies, e.g., of several classes, preferably, including IgG, IgA and IgM. A polyclonal antibody mixture may be advantageously used. Using defined antibodies such as recombinant antibodies may be advantageous if method of the invention is used in the context of routine quality control.

Depending on the selection of the antibody used, immune responses induced by different Ig classes can be evaluated, for example, by performing FcR blocking experiments, e.g., depending on the pathogen, and specific immunomodulation can be assessed. The skilled person is able to choose a suitable “bead” to be used in the context of the invention. The bead should be capable of being phagocytosed by the immune effector cell. To this end, preferably, the bead has a size of 30 nm-8 μm, such as 0.1-6.4 μm, preferably 0.5-2 μm, e.g., about 1 μm. In studies of phagocytosis by mouse peritoneal macrophages or human blood granulocytes, maximal phagocytosis was observed for an intermediate particle size (about 1.7 μm). With regard to uptake of 0.5-8 μm polystyrene microspheres by human blood neutrophils and leukocytes, it was reported that phagocytosis decreased with increasing particle size (Champion et al. 2008. For macrophages, the optimal size appears to be 0.5-2 μm (Pacheco et al. 2013, PLoS One 8(4):e60989).

The bead is an artificial body, preferably a solid body. Use of the beads in the method of the invention avoids the use of a virus, which is more difficult to handle and to standardize, and, furthermore, may be pathogenic. Thus, the bead is not a virus, pseudovirus or virus-like particle. In contrast to virus-like particles, the bead has the advantage of easier standardization. The bead may, e.g., be a latex bead, agarose bead, glass bead and gold bead, or a mixture thereof, preferably a latex bead. A bead may e.g., comprise different layers, i.e. it may be coated with a material different to the core of the bead. Typically, a plurality of beads of one kind is used.

The bead may also be labelled with a detectable marker, e.g. it may be fluorescent. If the bead is labelled, e.g. fluorescent, or if the bead is fluorescent per se, this has the advantage that phagocytosis of the beads into the effector cell can be easily monitored. This is however not required, e.g., in embodiments of the method in which cytokine production is assessed as a measure of potency. In a preferred embodiment, the bead is a latex bead of 30 nm to 6.4 μm size, e.g., 0.5-2 μm, e.g. a fluorescent latex bead of 30 nm to 6.4 μm size, e.g., 0.5-2 μm.

According to the invention, the bead is coated with the antigen, i.e., it comprises the antigen on its surface, or, in other words, it is surrounded by a layer comprising the antigen. While the surface must not be covered in its entirety with the antigen, it is preferred that the antigen is present in sufficient density to enable the formation of an immune complex capable of inducing a measurable response if bound to the FcR on the immune effector cell. This may also be achieved if the respective density is reached only in local patches on the surface.

The antigen is associated with the bead. Said association may be a non-covalent association, or a covalent linkage. Coated beads may be commercially available, or prepared, e.g., freshly prepared. Usually it is not important to maintain a certain orientation of the antigen on the bead. Thus the antigen may be associated in a random orientation. Alternatively, one may consider to ensure that the epitope recognized by the antibody is facing outward on the bead. This can be achieved e.g. by using an appropriate linker chemistry.

The bead can be coated with a single antigen. Alternatively, the bead can be coated with a plurality of different antigens. These may be different antigens from the same pathogen, or antigens from different pathogens, e.g., pathogens that are known to occur in co-infections, e.g., co-infections of the lung.

In one embodiment, the method of the invention thus further comprises preparing a bead coated with the antigen by incubating the bead with the antigen under conditions allowing association with the bead. Preferably, the antigen is covalently linked to the bead, e.g., via a carboxylate modification of the bead. Methods for covalent linkage of proteins, such as antigens, to beads, e.g., via carboxylate modification, are known in the art, and modified beads suitable for covalent linkage of proteins are commercially available. For example, 2*10⁸ beads, e.g., carboxylate modified latex beads, after washing with MES/EDAC (2-(N-morpholino) ethanesulfonic acid/1-(3-Dimethylaminopropyl)-3-ethylcarbodiimide) buffer, can be incubated with antigen, e.g., reSARS-CoV-2 spike protein, at a concentration such as 5 μg/mL under conditions suitable for linkage, e.g., for 30 min-24 h at 0-37° C., e.g., 1-4 h, e.g., 2 h at 37° C., preferably under agitation, e.g., on a shaker. An exemplary detailed protocol is provided in the example below.

The antigen may alternatively be non-covalently coated on the beads, e.g., by incubation of beads with a surface suitable for association of proteins, for 30 min-24 h at 0-37° C., e.g., 1-4 h, e.g., 2 h at 37° C.

Typically, free binding sites on the bead are blocked after incubating the bead with the antigen by an incubation with a protein different from the antigen, such as bovine serum albumin (BSA) (e.g., 0.1-2% BSA (w/v)). Alternatively, excess antigen may be used to prevent reactive free binding sites on the bead.

The method of the invention may further comprise preparing the bead coated with an antigen and an antibody specifically bound to said antigen. Thus, immune complexes are formed on the bead. This may be done by incubating the bead coated with the antigen with antibodies to said antigen. The preparation of immune complexes may e.g., be carried out with 1*10⁸ beads, e.g., latex beads, and about 5 μg/mL antibody, e.g., anti-SARS-CoV-2 Ig, or about 400 μg/mL convalescent plasma under conditions suitable for binding, e.g., for 30 min-24 h at 0-37° C., e.g., 45 min-1 h at 37° C.

Before the bead coated with an antigen and an antibody specifically bound to said antigen is employed in step b), it is advantageously washed, e.g., to avoid interference in the assay due to excess antibodies and/or immune complexes that may have detached from the bead.

The Immunoglobulin Test Composition

In step b) of the method of the invention, said bead (coated with an antigen and an antibody specifically bound to said antigen) is contacted with an immunoglobulin test composition and with an immune effector cell expressing at least one Fc-Receptor (FcR).

As mentioned, functional characterization and quality control are particularly important in the case of polyclonal and/or plasma-derived immunoglobulin test composition. Therefore, preferably, the immunoglobulin test composition is a polyclonal and/or plasma-derived immunoglobulin test composition. Typically, analysis of the potency of an immunoglobulin test composition is of highest interest if the immunoglobulins are of human origin. Thus, preferably, the test composition is a polyclonal immunoglobulin composition of human origin. Notably, plasma-derived immunoglobulin compositions for therapeutic use can be characterized and/or quality controlled by use of the method according to the invention.

The immunoglobulin test composition may be derived from human blood, plasma or serum. Typically, the immunoglobulins are derived from human plasma, preferably, polyclonal immunoglobulins derived from human plasma. If not specified in more detail, throughout this specification the term “plasma-derived immunoglobulin” means that the immunoglobulin is obtained directly or indirectly from human blood, e.g. from a blood donation, a plasma donation, or blood serum, most typically from a blood plasma donation.

The immunoglobulin test composition used in the context of the invention may comprise immunoglobulin of IgG, IgM and/or IgA class. Preferably, it comprises all three classes. It may also comprise IgD and/or IgE, typically, in low amounts (i.e. less than one percent, more particularly less than 0.5% of the total amount of immunoglobulins). For example, it may comprise only IgG, only IgM, or only IgA. It may also comprise IgG and IgM, but no significant amounts of IgA. It may also comprise IgG and IgA, but no significant amounts of IgM. Alternatively, it may comprise IgM and IgA, but no significant amounts of IgG. While the method according to the present invention is suited to measure effects of all immunoglobulin classes, it is particularly suited to measure effects of IgA. Therefore, the immunoglobulin test composition is particularly suited for testing immunoglobulin compositions comprising IgA. Therefore, preferably, the immunoglobulin test composition comprises IgG, IgM and IgA. Preferably, the immunoglobulin test composition is enriched in IgM, and comprises at least about 5% IgM, more preferably, at least about 10% IgM, more preferably at least about 18% IgM. It can thus also be designated IgM concentrate or IgM-containing immunoglobulin composition. Such compositions are sometimes also referred to as IVIG-M, or if they are further enriched for IgA, as IVIG-AM.

Throughout the application, antibody percentages are provided as weight/total immunoglobulin weight (w/total immunoglobulin w or w/total antibody w), i.e., antibody percentages of different classes IgG, IgA and IgM add up to 100%. Amounts of immunoglobulins indicated herein may be easily determined according to methods known in the art, for example using nephelometry in accordance with the European Pharmacopoeia 8.0, 2.7.1 (Siemens BN Prospec® System). The immunoglobulin test composition may or may not comprise IgD and IgE. Typically it does not comprise IgD and/or IgE in an amount exceeding 1% by weight.

The immunoglobulin test composition of the invention may e.g., comprise 5-90% IgM, optionally, 15-30% IgM, e.g., about 18-23% IgM.

It may alternatively, or, preferably, additionally, comprise 5-90% IgA, optionally, 15-25% IgA, e.g., about 21% IgA. It may further comprise 5-100% IgG, optionally, 40-80% IgG, e.g., about 50-60% IgG. In a preferred embodiment, it comprises about 50-60% IgG, about 18-25% IgM and about 15-25% IgA.

In a preferred embodiment, the immunoglobulin test composition comprises at least 30 g/L immunoglobulins, i.e., a higher concentration than blood, serum or plasma from healthy human donors. It can, therefore, also be designated an immunoglobulin concentrate. The immunoglobulin test composition preferably comprises at least 40 g/L immunoglobulins, e.g., g/L immunoglobulins or about 49-50 g/L. It does not contain any cells, and has typically been purified to contain, essentially, the immunoglobulins and a buffer suitable for administration to a human subject. Preferably, it does not comprise substantial amounts of complement factors or any complement factors. Typically, it is an aqueous solution. The immunoglobulins may be, e.g., at least 95% or at least 98% or at least 99% of the total protein (w/w).

In this case, the immunoglobulin test composition is derived from a plurality of donors. A composition derived from a plurality of donors has the advantage that it contains a mixture of immunoglobulins developed against pathogens to which a broad population of donors has been exposed and that it will be less dependent on individual exposure to antigens by single donors. A large population of donors will decrease the variation between different batches. Thus, in the context of the present invention, a plurality of donors preferably means from 2 donors to 10 000 donors, preferably 20 to 10 000 donors, more preferably 100 to 10 000 donors or still more preferably 500 to 8000. One donor can provide more than one donations. For example, the immunoglobulin test composition of interest in the present invention can derived from at least 500 donations or, preferably, at least 1000 donations. However, one of the advantages of the present invention is that it may be used to identify potency differences between different product batches, e.g. products derived from different donors or donors from different geographic regions.

A preferred immunoglobulin test composition derived from a plurality of donors comprises at least 30 g/L immunoglobulins and is derived from a plurality of human donors and comprises at least 5%, at least 10% or, preferably, at least 15% IgM and/or IgA. It may also comprise at least 20% IgM and/or IgA. It may comprise at least 5%, at least 10% or, preferably, at least 15% IgM and IgA, respectively (i.e., for example at least 15% IgM and at least 15% IgA). It is particularly preferred that it comprises at least 20% IgM and at least 20% IgA.

A preferred immunoglobulin test composition to be used in the context of the present invention comprises purified human immunoglobulin of the IgG, IgM and IgA classes in relative concentrations of about 18-28% IgM, about 16-26% IgA and about 51-61% IgG, e.g., about 23% IgM, about 21% IgA and about 56% IgG and less than 1% of other immunoglobulins (e.g., trimodulin produced by Biotest AG). Examples for other immunoglobulin test compositions of interest include Pentaglobin (Biotest AG) containing 72% IgG and enriched in IgM (12%) and IgA (16%), which has been treated with beta-propiolactone, and IgAbulin (Immuno AG, Vienna), which mainly comprises IgA (73%), IgG (26%) and small amount of IgM (1%)

Typically, the immunoglobulin test composition shall be suitable for pharmaceutical use, more preferably approved for pharmaceutical use. Typically, the immunoglobulin test composition is purified, e.g. it contains at least 95% immunoglobulins by weight of total protein, more typically at least 98%. Also typically, the immunoglobulin test composition is virus-safe, e.g. the manufacturing should comprise at least two effective steps (more than 3 log 10 reduction, preferably more than 4 log 10 reduction) for inactivation of enveloped viruses and at least one effective step for inactivation of non-enveloped viruses. Such steps are known to the skilled person. For example, such steps may be selected from the group comprising precipitation (in particular ethanol precipitation), nanofiltration, pasteurization, octanoic acid precipitation, UV-treatment, low pH treatment, solvent-detergent treatment, and chromatography.

In one embodiment, the immunoglobulin test composition is chemically modified, e.g. by treatment with beta-propiolactone. In another embodiment, the immunoglobulin test composition is not intentionally chemically modified. This means that it has not been treated with agents intended to lead to chemical modifications. Certain modifications may, however, occur during the preparation. Thus, the composition substantially does not comprise chemically modified immunoglobulin. Methods of preparation of immunoglobulin test composition are disclosed in WO 2011/131786 A2 and WO 2011/131787 A2, and in Examples 1 and 2 below. For example, in one embodiment, the immunoglobulin test composition does not comprise immunoglobulin modified with beta-propiolactone.

In an alternative embodiment, the immunoglobulin test composition is derived from a single subject. In this context, e.g., the inflammatory status of the patient can be assessed by the method of the invention. Alternatively, the immunoglobulin test composition derived from a subject may also be assessed for its potency for treatment of a disease caused by a pathogen expressing the antigen used (e.g. as convalescent plasma or an immunoglobulin composition derived thereof). In this embodiment, the immunoglobulin test composition may be plasma or serum, or it may be derived therefrom. For example, it may be plasma. As a specific example, the immunoglobulin test composition may e.g., be derived from a convalescent patient who had a disease associated with the antigen, e.g., COVID-19 wherein the antigen is SARS-CoV-2 surface protein such as SARS-CoV-2 spike protein.

In general, it is not important if the immunoglobulin test composition comprises immunoglobulins capable of binding to the antigen coated on the beads. Therefore, the immunoglobulin test composition may or may not comprise immunoglobulins capable of specifically binding to the antigen coated on the beads. For example, if the antigen is SARS-CoV-2 spike protein, the immunoglobulin test composition may or may not comprise immunoglobulins to said protein. However, if the immunoglobulin test composition does comprise immunoglobulins capable of specific binding to the antigen coated on the beads, it should be made sure that the specific antibody used to bind to the antigen on the bead, prior to contacting with the immunoglobulin test composition, is used in sufficient amounts bind substantially to all available binding sites on the bead. This should be done to avoid an undesired influence of specifically binding immunoglobulins comprised in the test composition,

The immunoglobulin test composition further does not consist of immunoglobulins directed to a human Fc receptor, i.e., binding to it via the antigen-binding sites, and, optionally, does not comprise such immunoglobulins. More specifically, the immunoglobulin test composition does not comprise such immunoglobulins capable of specific binding to a human Fc receptor in an amount that would impair the test result. Typically, the immunoglobulin test composition comprises immunoglobulins that are directed to a plurality of antigens. Characterization or knowledge of said antigens is not required, and, typically, the antigens will be uncharacterized.

Further, in any case, the immunoglobulin test composition preferably does not comprise complement to avoid any modulation by complement. The assay however also works in the presence of complement. Thus, if said modulation is of interest, the test composition may comprise complement.

For characterisation and quality control, e.g., for determination of the potency, the immunoglobulin test composition may be serially diluted or titrated. Preferably, the immunoglobulin concentration is in the range of 0.005-15 g/L. It is noted that this concentration, as other concentrations cited herein, if not specifically disclosed otherwise, relates to the mixture of step b). The preferred concentrations disclosed herein are adapted to have optimal results if used in combination with each other. Of course, the skilled person will be able to modify said concentrations and adapt them, e.g., if one of the components is present in a different concentration or has a different biological activity.

Preferably, step b) is carried out with different concentrations of the immunoglobulin test composition, e.g. at least 2, 3, 4, or 5 different dilutions. Preferably, at least 3 different concentrations are employed, more preferably 4 or 5. The skilled person will be able to determine suitable concentrations. The different concentrations are typically prepared by dilution, e.g., 1:1 dilutions, 1:2 dilutions or 1:10 dilutions.

To reduce variation of measurement, each concentration may be tested in multiple (e.g. 2, 3 or 4) samples.

Preferably, the concentration of beads and immune effector cells is constant when different concentrations of the immunoglobulin test composition are tested.

Advantageously, the method can be carried out in a suitable array format, such as in a multi-well plate, e.g. a 96-well plate.

As controls, physiologic buffer (e.g., D-PBS or PBS or media or formulation buffer) may be tested instead of immunoglobulin test composition, preferably the same buffer in which the immunoglobulin test concentration is otherwise added in step b) of the method according to the invention. Such control may be taken to determine the value for baseline lysis in absence of immunoglobulin test composition.

The Immune Effector Cell

A suitable immune effector cell may be chosen be the skilled person. A suitable immune effector cell expresses at least one class of Fc receptor, preferably, at least two or at least three classes of Fc receptors. Preferably, the immune effector cell expresses receptors for both IgG (CD16, CD32 and/or CD64) and IgA (e.g., CD89). It may, e.g., express CD16, CD32, CD64 and CD89. The immune effector cell should express the Fc receptors when used in the method according to the invention, it is not necessary that the expression of the Fc receptor occurs under any culture conditions. Expressing in the present context means that the cell presents a sufficient amount of Fc receptor in order to respond to binding of an antibody or immunoglobulin Fc-part. Preferably, the response is measurable in dose-dependent manner.

Accordingly, the immune effector cell preferably is selected from the group comprising neutrophils, eosinophils, monocytes, macrophages, and dendritic cells (DC). Preferably, the cell is a human cell, as human immunoglobulin compositions are typically tested.

In particular, the immune effector cell can be a granulocytic cell such as a neutrophil or eosinophil. Neutrophils are preferred. This includes promyelocytic cells, in particular, promyelocytic cells matured with a suitable agent (such as DMSO, DMF, retinoic acid, or 1α,25 dihydroxyvitamin D2, preferably, DMSO in case of HL60 cells, and LPS in case of dendritic cells) or comparable components suitable for differentiation of the used cell line, the skilled person will know. For example, the neutrophil can be a HL60 cell, preferably, a differentiated HL60 cell. HL-60 cells may e.g., be cultivated with DMSO (e.g., 1-2% for 3-7 days, e.g., 1.3% DMSO 3-5 days).

Neutrophils such as HL-60 cells can be additionally stimulated with LPS (e.g., 10-1000 ng/mL, preferably, about 500 ng/mL) for about 12-60 hours, e.g., 24-48 hours before use in the assay of the invention.

The HL60 cells employed express CD16, CD32, CD64 and CD89. CD16 is reported to be expressed by a lower percentage of cells, e.g., 20-40%, while the other Fc receptors analysed are reportedly expressed by more than 90% of cells, or substantially all of the cells. LPS leads to a reduced expression of CD16, while expression of the other analysed Fc receptors is increased by stimulation with LPS for 24 h or more.

Although not much is known about the function of neutrophils in virus-induced diseases, there is evidence for involvement of these cells in influenza, severe acute respiratory syndrome-related coronavirus (SARS-CoV-1) and Middle East respiratory syndrome coronavirus (MERS-CoV) infection. If neutrophils are in this context beneficial or detrimental is under discussion (Camp and Jonsson 2017. Front. Immunol. 8). In accordance with data from related coronaviruses, neutrophil infiltration was shown in clinical studies with COVID-19 patients (Huang et al. 2020. The Lancet 395, 497-506).

Alternatively, the immune effector cell can also be a monocyte, e.g. a THP-1 or U937 cell. Dendritic cells in immature or mature form may also be used. Such cells can be used in the assay of the invention after stimulation with suitable components known in the art, e.g., with PMA (phorbol 12-myristate 13-acetate) or LPS, or without stimulation. Ackermann et al., 2011 (J Immunol Methods 366(1-2):8-19) teach an assay for determining the phagocytic activity of clinical antibody samples, wherein a monocytic cell line expressing Fc receptors, THP-1 cells, is contacted with antigen-coated beads to which antibodies are added to allow for formation of immune complexes. After incubation, phagocytosis of beads or cytokine production is determined. Blocking antibodies are used in some tests to elucidate contribution of different Fc receptors to the response. In contrast to the method of the present invention, Ackermann et al. are however interested in the characteristics of the antibodies used for forming the immune complexes. There is thus no immunoglobulin test composition that is tested for its immunomodulatory potency. However, under many conditions, it is preferable not to use a monocyte cell, in particular not a THP-1 or U937 cell. Monocytic cells are typically adherent cells that are not advantageous to use in a routine assay. Preferably, the cells used are non-adherent cells.

Generally, it is possible to use primary immune effector cells, which can be prepared, e.g., according to methods known in the art. Alternatively, cell lines may be used, e.g., those mentioned above.

In the method of the invention, contacting of the bead (coated with an antigen and an antibody specifically bound to said antigen) with the immunoglobulin test composition and with the immune effector cell expressing at least one Fc-Receptor (FcR) in step b) of the method of the invention can be in either order or substantially at the same time.

In step b), the bead (coated with an antigen and an antibody specifically bound to said antigen) may first be contacted with the immunoglobulin test composition to form a mixture, and the resulting mixture may then be contacted with the immune effector cell. In this case, it is not significant how long the mixture is incubated before contact with the immune effector cell.

Alternatively, in step b), the immune effector cell is first contacted with the immunoglobulin test composition to form a mixture, and the resulting mixture is then contacted with the bead (coated with an antigen and an antibody specifically bound to said antigen). Such a preincubation, if it is carried out for a significant time, e.g., more than 5 min, more than 10 min, more than 15 min or more than 30 min, e.g., about an hour, may increase the immunomodulatory effect of the immunoglobulin test composition. However, a more physiologic approach is preferred that mimics the situation in a patient where the immunomodulatory composition is not preincubated with the immune effector cells before stimulation, e.g., with immune complexes.

Thus, in step b), the immune effector cell may also be first contacted with the bead (coated with an antigen and an antibody specifically bound to said antigen) to form a mixture, and the resulting mixture may then contacted with the immunoglobulin test composition.

Preferably, the contacting with immune effector cells and immunoglobulin test composition in step b) is substantially simultaneously, i.e., within at most 30 min, within at most 15 min or, optimally, within at most 5 min. For example, the immune effector cells can be present in the container, e.g., in the wells of a tissue culture plate, first, and then the beads and the immunoglobulin test composition added substantially at the same time.

In one embodiment, the invention also discloses a method for assessing the immune status of a subject, comprising
a) providing a bead coated with an antigen and an antibody specifically bound to said antigen,
b) contacting said bead with an immunoglobulin composition and with an immune effector cell expressing at least one Fc-Receptor (FcR), wherein said immune effector cell is derived from the subject, and
c) determining an effector function of the immune effector cell.

In particular, said method can be used to determine the capability of the immune effector cell of the subject for phagocytosis and/or, in particular, for performing the effector function determined in dependency from the immunoglobulin composition. This allows conclusions on the capability of the immune effector cell to react to immunomodulation, and thus, on the inflammatory status of the subject. Additionally the method can be used to test the plasma of said subject. Therefore, it is possible to test the success of the therapy before the therapy in vitro. The subject may be a patient undergoing an undesired and/or inappropriately strong immune response, e.g., in the context of sepsis or COVID-19. The components used may be used as described herein otherwise for the method of the invention. The immunoglobulin composition used in this context preferably has a known immunomodulatory potency.

The Effector Function and Determination of Potency

In step c), an effector function of the immune effector cell is determined.

The effector function is selected from the group consisting of cytokine production, phagocytosis of the beads, modulation of surface markers, NETose, ROS release and degranulation.

An effector function that can easily and reproducible be determined, also in an automated assay, is cytokine production. Cytokine production can be cytokine secretion, which can, e.g., be determined via an ELISA or multiplex assay or ELISPOT assay, preferably, ELISA. Alternatively, cytokine production can be determined by qPCR or by intracellular FACS.

If the effector function is determined using a cytokine, the cytokine may be chosen as deemed appropriate, also depending on the cytokines secreted by the particular immune effector cell. Preferably, a suitable cytokine shows a strong dose response if contacted with beads coated with the antigen-antibody complex and, more importantly, if subsequently contacted with the immunoglobulin test composition to be analyzed. Depending on the cell type of the immune effector cell, the cytokine may be a pro-inflammatory cytokine, e.g., TNF-alpha, GM-CSF, RANTES, GRO-alpha, MIP1 alpha, MIP1beta, MCP-1, MIF, M-CSF, ICAM-1, Serpin E-1, IL-1alpha, IL-1beta, IL-2, IL-3, IL-6, IL-7, IL-8, IL-9, IL-12, IL-17, IL-21, IL-22, IL-23, IFN-gamma, preferably, IL-8, IL-6, TNF-alpha, and/or IFN-gamma. The cytokine may also be anti-inflammatory, e.g. IL-1ra, IL-4, IL-5, IL-10, IL-11, IL-13. Notably, certain cytokines, e.g., IL-10, may be considered to be pro-inflammatory or anti-inflammatory depending on the circumstances. E.g. IFN-alpha or TGF-beta may also be used in the method of the invention.

For example, the inventors could show that, using neutrophils such as HL60 cells as immune effector cells, the cytokine IL-8 can be advantageously determined, e.g., secretion of IL-8. If the immune effector cell is a macrophage or monocyte, for example, IL-6 can also advantageously be determined.

Multiple cytokines may be determined, either separately or in parallel (e.g. to improve the sensitivity of the readout), but that is not required.

Furthermore, in the context of the present invention, it could be shown that determination of the potency of an immunoglobulin test composition by determining inflammatory cytokine production of the immune effector cell, e.g., IL-8 secretion, is advantageous. In particular, the inventors could show that it is advantageous for testing compositions comprising significant amounts of IgA (e.g., at least 10%, at least 15% or, preferably, at least 20%), and optionally, significant amounts of IgM (e.g., at least 10%, at least 15% or, preferably, at least 20%). In particular, IL-8 production, e.g., IL-8 secretion, was shown to be reduced significantly more by IgA-containing immunoglobulin test compositions such as IVIG-AM compared to immunoglobulin test compositions not comprising significant amounts of IgA. Such immunoglobulin compositions that may be tested, and, optionally, standardized, with the method of the invention may be, e.g., for use in treatment in the respiratory tract, where IgA plays a particular role, e.g., inflammation associated with COVID-19 or sCAP or influenza. Thus, compositions for use in such indications may be preferably analysed by determining inflammatory cytokine production of the immune effector cell, e.g., IL-8 secretion.

Based on this readout, the assay of the invention was able to show an improved immunomodulatory potency of immunoglobulin test compositions comprising IgA and, optionally, IgM compared to immunoglobulin test compositions comprising substantially only IgG. This finding is in line with previous preclinical and clinical findings. In this context, the beads are preferably coated with an antigen associated with the relevant pathogen of interest, e.g., an influenza HA antigen or SARS-CoV-2 spike protein and bound by antibodies from convalescent plasma containing antibodies against said antigen.

The effector function may alternatively (or additionally) be modulation of a surface marker selected from the group comprising CD14, CD11 b, CD35, CD86 and FcR, e.g., CD89, CD64, CD32 and CD16. Modulation of a surface marker may be detected on the level of surface expression, e.g., per flow cytometric analysis, or on the level of gene expression.

Phagocytosis of beads, e.g., fluorescent beads, which, in particular for fluorescent beads, can easily be tested by flow cytometric analysis, can also be determined as the effector function. This was shown to be dependent on the IgG and IgA component of the antibodies used for forming the immune complex on the bead, with no significant additional effect of the IgM component (data not shown). Upon incubation with different immunoglobulin test compositions, phagocytosis was reduced. No significant difference between IVIG and IVIG-AM enriched for IgM and IgA could be detected with regard to phagocytosis. Thus, this effector function is preferably used if potency of the IgG component of an immunoglobulin test composition comprising several antibody classes, or of immunoglobulin composition comprising essentially IgG is to be analysed. It may also be used to analyze the IgG component in an IVIG-AM selectively.

Additionally and/or alternatively to cytokine secretion and phagocytosis other effector functions directly mediated by the bead stimulation of the cells can be used. Depending on the FcR bearing cell type used, the skilled person is able to determine effector functions based on the current knowledge and standard protocols (e.g., Aleyd et al., 2014, J Immunol 192:2374-2383; Aleyd et al, 2016, J Immunol 197:4552-4559; Heineke et al., 2017, Eur. J. Immunol. 47: 1835-1845; Seyrantepe et al. 2010, JBC 285(1): 206-215; von der Steen et al., 2009, Gastroenterology 137:2018-2029). These can include as example for neutrophils (or neutrophil-like cells) NET (neutrophil extracellular traps) release, NETose, ROS (reactive oxygen species) release, cell migration, degranulation or measurements of changed gene expression.

Preferably, the effector function is compared with the effector function of a control test carried out without contacting the bead coated with an antigen and an antibody specifically bound to said antigen (or irrelevant antigen) with the immunoglobulin test composition to determine a change in effector function, i.e., in the absence of an immunoglobulin test composition. In other words, for the control, the method of the invention is carried out without in step b) contacting the bead of step a) with an immunoglobulin test composition. Thus, a maximal effector function of the immune effector cell can be determined. The “control potency” determined based on said control is considered to be 0% potency.

Further, it is advantageous to compare the potency of the immunoglobulin test composition(s) being tested, i.e., of the sample(s), to the potency of a standard immunoglobulin composition, e.g., a reference standard immunoglobulin composition. Such reference standard may be e.g., a sample of a public reference standard such as available for certain immunoglobulin preparations e.g. from CBER (Center for Biologics Evaluation and Research, USA) or EDQM (European Directorate for the Quality of Medicines and Healthcare, Council of Europe, Strasbourg, France). Alternatively, the reference standard may be an internal reference standard. E.g. it could be an internal standard of an IgA and IgM-containing immunoglobulin composition. The ratio of the potency of the immunoglobulin test composition to the potency of the standard immunoglobulin composition is the relative potency. Potency of the standard, e.g. an IgA and IgM-containing composition reference standard, is set to 100%. The standard immunoglobulin composition preferably is a standard IgM and IgA containing immunoglobulin composition.

One aspect of characterisation and quality control is determination of potency or effectiveness, which is a measure of drug activity expressed in terms of the amount required to produce an effect of given intensity. A highly potent drug evokes a given response at low concentrations, while a drug of lower potency evokes the same response only at higher concentrations. The potency depends on both the affinity and efficacy of the active agent. Affinity is how well a drug can bind to a target. Efficacy is the relationship between target occupancy and the ability to initiate a response.

In the context of the present invention, potency is a measure of the amount required to produce an effect of a given intensity, e.g., an immunomodulatory effect in the treatment of an inflammation, for example in the context of COVID-19. However, even if potency is determined using a SARS-CoV-2 antigen and an antibody capable of binding said antigen, the method of the invention is not limited to determination of the potency against inflammation, for example in the context of COVID-19, but can further be used as a measure of general immunomodulatory potency, e.g., in the treatment of other diseases causing excessive inflammation. Moreover, the potency as measured according to the present invention can also be taken as an indicator of the general functional integrity of the immunoglobulin test composition. E.g. if immunoglobulins in the composition have been denatured or partially denatured, the immunomodulatory capability is affected as well as binding to specific antigens. However, in a narrow sense, the term potency according to the present invention means the ability of the immunoglobulin test composition for immunomodulation. Thus, preferably, the potency is immunomodulatory potency. Immunomodulation in the context of the invention typically is inhibition of the immune response, in particular, inhibition of an excessive immune response.

The inhibition of the effector function by the immunoglobulin test composition that is determined by the method of the invention is positively correlated to the potency of the immunoglobulin test composition.

Advantageously, the inhibition of the effector function by the immunoglobulin test composition is also positively correlated to the efficiency of the immunoglobulin test composition in treatment of inflammation. The inflammation may be acute respiratory syndrome. The inflammation may also be sepsis, which may be associated with a cytokine storm. In one embodiment, the inflammation is in the context of COVID-19. The inflammation may alternatively be in the context of severe community acquired pneumonia (sCAP), which may be caused by a bacterial and/or viral infection, e.g., a pneumococcal infection. The inflammation may also be due to autoimmunity or allergy.

A dose response curve may be determined based on the samples with different concentrations of the immunoglobulin composition. The analysis of potency may then carried out based on a shift of the dose-response curve, e.g., using parallel line assessment.

If each concentration is tested in multiple samples, an average value may be calculated and used as a basis for the dose-response curve. A mean value or weighted average may be determined from the parallel samples. Alternatively, curve-fitting can be carried out with all single values.

Testing of multiple samples, typically, in parallel, allows to reduce variability of test results based on variation between single samples, e.g. due to variation in pipetting.

Potency is calculated, preferably, by comparing dose-response curves of the immunoglobulin composition to be tested with the dose-response curve of a suitable reference standard. For example, parallel line assessment and a 4 or 5 Parameter logistic fit can be used (e.g. using Software PLA 3.0, Stegmann Systems GmbH).

The invention thus provides a method for characterisation and quality control of an immunoglobulin test composition comprising, e.g., immunoglobulins derived from a plurality of human donors, comprising testing potency of the immunoglobulin test composition by the method of the invention, as described herein.

The method for characterisation and quality control, or the potency assay of the invention can be performed on the drug substance, typically, a solution more highly concentrated that the desired final drug product, i.e., the immunoglobulin test composition to be tested can be the drug substance. The drug substance is a bulk concentrate or bulk intermediate comprising the active pharmaceutical ingredient, and it is not yet packaged in single units, but used to formulate the drug product, i.e., the dosage form or finished product which comprises the drug substance, usually further comprising excipients. The method of the invention can also be carried out on the drug product, which may optionally already be packaged in a primary packaging. Alternatively or additionally, intermediates in the production process, in process samples, development samples and stability samples can be tested by the method of the invention, which allows for an early selection of production batches or parts thereof, e.g., pooled compositions leading to a desired potency of the drug product, e.g., a potency of 50-200% relative to a standard immunoglobulin composition such as a standard IgM and/or IgA-containing composition.

The desired potency of the drug product may be, e.g., a potency of 50-200% or 70-150% or 80-120% relative to a standard immunoglobulin composition such as a standard IgM and/or IgA-composition. Preferably, the desired potency is obtained at a defined concentration of the immunoglobulin test composition, e.g., 50-70 g/L or 40-60 g/L.

Methods of Preparation

The invention also provides a method for preparing a standardized immunoglobulin composition comprising at least 30 g/L immunoglobulins derived from a plurality of human donors, comprising
i. pooling plasma or serum derived from a plurality of human donors to provide a pool;
ii. isolating and concentrating immunoglobulins from the pool to produce an immunoglobulin test composition;
iii. characterising or testing quality of the immunoglobulin test composition of ii) by the method of the invention, wherein said immunoglobulin test composition is optionally discarded if the relative potency of said immunoglobulin test composition is not in a predetermined range, e.g., of 50-200%, of the potency of the standard immunoglobulin composition; and
iv. optionally, adapting the potency of the immunoglobulin test composition to a desired potency (e.g., 50-200% of the potency of the standard immunoglobulin composition); and/or
v. optionally, packaging an amount of the immunoglobulin test composition, e.g., an amount having a desired potency.

The predetermined range may also be, e.g., 80-120% of the potency of the standard immunoglobulin composition.

Adapting the potency may e.g., be carried out if the relative potency of said immunoglobulin test composition is in the predetermined range, e.g., of 50-200%, of the potency of the standard immunoglobulin composition. In that case, it may still be of interest to prepare a product for which the batches all have essentially the same potency, e.g., 80-120%, 90-110% or about 100% of the potency of the standard immunoglobulin composition.

Preferably, no adaptation of the potency is required.

Packaging at least comprises packaging in a secondary packing (e.g., cardboard boxes) and adding instructions for use. Packaging of the final step consists of these steps if the testing of step iii. is performed on one or more samples from the drug product which is already filled into a primary packaging. Multi-unit packaging may still be added. Optionally, packaging further comprises packaging in a primary packaging, e.g., in vials, bottles, syringes, plastic backs for infusion etc., in particular, if the testing of step iii is performed on the drug substance. Testing can also be performed on drug substance which has already been diluted to the final concentration, but not yet packaged.

The immunoglobulin containing composition can be stored as deemed appropriate by the skilled person. For example, often, plasma or serum are stored before or after pooling in order to perform tests on the donors or the donations, e.g., to prevent contaminations with pathogens. Of course, the final product can also be stored, as described.

Kits and Compositions

In another aspect, the invention also provides a kit for carrying out the method of the invention, comprising
the bead, the antigen and the antibody specific to said antigen, and
a standard immunoglobulin composition comprising at least 30 g/L immunoglobulins derived from a plurality of human donors, optionally, an IgM and/or IgA containing immunoglobulin composition.

The bead may optionally already be coated with the antigen. Optionally, the bead is already coated with the antigen and the antibody is specifically bound to said antigen.

Alternatively, the bead, the antigen and the antibody are present in separate containers. In this case, the kit preferably comprises instructions to prepare a bead coated with the antigen and the antibody specifically bound to said antigen.

The standard immunoglobulin composition in the kit may comprise IgG, IgM and/or IgA, wherein, preferably, the immunoglobulin composition comprises about 50-60% IgG, about 20-25% IgM and about 18-23% IgA, e.g., about 56% IgG, about 23% IgM and about 21% IgA.

A kit of the invention optionally further comprises immune effector cells capable of expressing FcR, e.g., as described herein, e.g., HL60 cells, THP-1 or U937, preferably, HL-60 cells.

The invention also provides a composition comprising a human immunoglobulin test composition comprising at least 30 g/L immunoglobulins derived from a plurality of donors and a bead coated with an antigen and an antibody specifically bound to said antigen. Said composition may optionally comprise immune effector cells expressing FcR. In one embodiment, said human immunoglobulin test composition comprises about 50-60% IgG, about 20-25% IgM and about 18-23% IgA, e.g., about 23% IgM, about 56% IgG and about 21% IgA. Such compositions are, e.g., obtained when the method of the invention is carried out.

The invention further provides an immunoglobulin composition comprising immunoglobulins derived from a plurality of human donors standardized to a desired potency, preferably, a relative potency of 50-200% or 80-120%, by the method of determining the potency of an immunoglobulin test composition described herein, wherein the immunoglobulin composition preferably is obtainable from the method for preparing a standardized immunoglobulin composition described herein. Preferably, a plurality of such standardized immunoglobulin composition, in particular, a plurality of charges or batches of such standardized immunoglobulin composition are provided, which comprise immunoglobulins derived from a plurality of donors as described herein, wherein all charges or batches have a potency in the same range determined by the method of the invention.

The tested and, optionally, standardized immunoglobulin composition is useful for immunomodulation, particularly, in the treatment of inflammation, as described herein, e.g., for treatment of excessive inflammation in the context of COVID-19. It is particularly advantageous if the tested immunoglobulin composition comprises IgM in a concentration of more than 12%, preferably, more than 18% and/or IgA in concentrations of more than 10%, preferably, more than 16% (e.g., trimodulin, Biotest AG).

The immunoglobulin composition to be tested may be intended for therapy, particularly for therapy of a human patient. Immunoglobulin compositions have neutralizing activities against infectious agents such as bacteria, viruses and their toxins, as well as immunomodulatory properties. E.g. the immunoglobulin composition tested may be for treatment of bacterial infections, e.g., in pneumococcal infection, diphtheria, pertussis, tetanus, botulism, staphylococcal infection, pseudomonas infection, or sepsis. E.g. the immunoglobulin composition tested may be for passive immunization for the prevention of measles, hepatitis A, hepatitis B, tetanus, varicella, rabies and vaccinia. E.g. the immunoglobulin composition tested may also be for treating viral infections, e.g. SARS-CoV-2, cytomegalovirus, parvovirus B19, and enterovirus infections, e.g., in immunocompromised patients. Immunoglobulin compositions may also be used in treating in toxic shock syndrome, Ebola virus, and refractory staphylococcal infections (Keller et al., 2000. Clin Microbiol Rev. 13(4): 602-614).

Immunodeficient patients, e.g., patients who do not generate antibodies in protective levels, such as patients with primary immunodeficiency, e.g., patients with IgG, IgA and/or IgM deficiencies, can particularly benefit from administration of the immunoglobulin composition for general prevention of infections (Perez et al. 2017, J Allergy Clin Immunol. 139:S1-46).

E.g. the immunoglobulin test composition tested may be for treatment of secondary immunodeficiencies. E.g. patients experiencing severe stress associated with trauma, extensive surgery, or intensive care have profound susceptibility to infection and develop a spectrum of immune deficiencies including cutaneous anergy, phagocytic dysfunction, hypogammaglobulinemia, and transiently impaired antibody function (Glinz et al., 1985, Intensive Care Med. 11:288-294). Administration of plasma-derived immunoglobulins, in particular, concentrated immunoglobulin compositions, can be beneficial.

The following examples and embodiments are intended to illustrate the scope of the invention, and do no limit the same. All references cited herein are fully incorporated herein by reference.

The invention e.g. provides the following embodiments:
1. A method for testing potency of an immunoglobulin test composition, the method comprising
a) providing a bead coated with an antigen and an antibody specifically bound to said antigen,
b) contacting said bead with said immunoglobulin test composition and with an immune effector cell expressing at least one Fc-Receptor (FcR), and
c) determining an effector function of the immune effector cell.

2. Use of a bead coated with an antigen and an antibody specifically bound to said antigen for testing potency of an immunoglobulin test composition.

3. The use of embodiment 2, comprising
a) providing a bead coated with an antigen and an antibody specifically bound to said antigen,
b) contacting said bead with said immunoglobulin test composition and with an immune effector cell expressing at least one FcR, and
c) determining an effector function of the immune effector cell.

4. The method of embodiment 1 or the use of any of embodiments 2-3, wherein the potency is determined based on the effector function of the immune cell.

5. The method of any of embodiments 1 or 4 or the use of any of embodiments 2-4, wherein the antigen is a viral surface protein.

6. The method of any of embodiments 1 or 4-5 or the use of any of embodiments 2-5, wherein the antigen is a viral spike protein.

7. The method of any of embodiments 1 or 4-6 or the use of any of embodiments 2-6, wherein the antigen is a viral nucleocapsid protein.

8. The method of any of embodiments 1 or 4-6 or the use of any of embodiments 2-6, wherein the antigen is a viral envelope protein.

9. The method of any of embodiments 1 or 4-6 or the use of any of embodiments 2-6, wherein the antigen is SARS-CoV-2 surface protein.

The method of embodiment 9 or the use of embodiment 9, wherein the antigen is SARS-CoV-2 spike protein.

11. The method of embodiment 10 or the use of embodiment 10, wherein the antigen is full length SARS-CoV-2 spike protein, optionally further including a His-tag.

12. The method of any of embodiments 1 or 4-11 or the use of any of embodiments 2-11, wherein the antigen is recombinant.

13. The method of any of embodiments 1 or 4-12 or the use of any of embodiments 2-12, wherein said antibody specifically bound to said antigen is an antibody from a patient who has been infected with a pathogen expressing said antigen, preferably, heat-inactivated plasma of a convalescent COVID-19 patient.

14. The method of any of embodiments 1 or 4-12 or the use of any of embodiments 2-12, wherein said antibody specifically bound to said antigen is recombinant antibody.

The method of any of embodiments 1 or 4-14 or the use of any of embodiments 2-14, wherein the bead is capable of being phagocytosed by the immune effector cell.

16. The method of any of embodiments 1 or 4-15 or the use of any of embodiments 2-15, wherein the bead is selected form the group consisting of latex beads, agarose beads, glass beads and gold beads.

17. The method of any of embodiments 1 or 4-16 or the use of any of embodiments 2-16, wherein the bead is a latex bead.

18. The method of any of embodiments 1 or 4-17 or the use of any of embodiments 2-15, wherein the bead has a size of 30 nm-6.4 μm, preferably 0.5-2 μm, e.g., about 1 μm.

19. The method of any of embodiments 1 or 4-18 or the use of any of embodiments 2-18, wherein the bead is fluorescent.

The method of any of embodiments 1 or 4-19 or the use of any of embodiments 2-19, further comprising preparing a bead coated with the antigen by incubating the bead with the antigen.

21. The method of any of embodiments 1 or 4-20 or the use of any of embodiments 2-20, wherein the antigen is covalently linked to the bead, optionally, via a carboxylate modification of the beads.

22. The method of any of embodiments 1 or 4-20 or the use of any of embodiments 2-20, wherein the antigen is non-covalently coated on the beads.

23. The method of any of embodiments 1 or 4-22 or the use of any of embodiments 2-22, wherein free binding sites on the bead are blocked after incubating a bead with the antigen e.g., with BSA.

24. The method of any of embodiments 1 or 4-23 or the use of any of embodiments 2-23, further comprising preparing the bead coated with an antigen and an antibody specifically bound to said antigen by incubating the bead coated with the antigen with antibodies to said antigen, wherein, preferably, the prepared bead is washed before step b).

25. The method of any of embodiments 1 or 4-24 or the use of any of embodiments 2-24, wherein the immunoglobulin test composition is a polyclonal immunoglobulin composition.

26. The method of any of embodiments 1 or 4-25 or the use of any of embodiments 2-25, wherein the immunoglobulin test composition comprises at least 30 g/L immunoglobulins and is derived from a plurality of human donors.

27. The method of any of embodiments 1 or 4-26 or the use of any of embodiments 2-26, wherein the immunoglobulin test composition comprises IgG, IgM and/or IgA, preferably, all three classes.

28. The method of any of embodiments 1 or 4-27 or the use of any of embodiments 2-27, wherein the immunoglobulin test composition comprises 5-90% (w/total antibody w) IgM, optionally, 15-30% IgM, e.g., about 23% IgM.

29. The method of any of embodiments 1 or 4-28 or the use of any of embodiments 2-28, wherein the immunoglobulin test composition comprises 5-90% (w/total antibody w) IgA, optionally, 15-25% IgA, e.g., about 21% IgA.

30. The method of any of embodiments 1 or 4-29 or the use of any of embodiments 2-29, wherein the immunoglobulin test composition comprises 5-100% (w/total antibody w) IgG, optionally, 40-80% IgG, e.g., about 50-60% IgG, wherein, preferably, the immunoglobulin test composition comprises about 50-60% IgG, about 20-25% IgM and about 20-25% IgA.

31. The method of any of embodiments 1 or 4-25 or 27 or the use of any of embodiments 2-25 or 27, wherein the immunoglobulin test composition is plasma or serum or derived therefrom, optionally, plasma.

32. The method of any of embodiments 1 or 4-25 or 27 or 31 or the use of any of embodiments 2-25 or 27 or 31, wherein the immunoglobulin test composition is derived from a convalescent patient who had a disease associated with the antigen, e.g., COVID-19 wherein the antigen is SARS-CoV-2 surface protein such as SARS-CoV-2 spike protein.

33. The method of any of embodiments 1 or 4-32 or the use of any of embodiments 2-32, wherein the immunoglobulin test composition does not comprise complement.

34. The method of any of embodiments 1 or 4-33 or the use of any of embodiments 2-33, wherein the immune effector cell expresses CD16, CD32, CD64 and CD89.

35. The method of any of embodiments 1 or 4-34 or the use of any of embodiments 2-34, wherein the immune effector cell is selected from the group comprising neutrophils, eosinophils, monocytes, macrophages, and DC.

36. The method of embodiment 35 or the use of embodiment 35, wherein the immune effector cell is a neutrophil.

37. The method of embodiment 36 or the use of embodiment 36, wherein the immune effector cell is a HL60 cell, preferably, a differentiated HL60 cell.

38. The method of embodiment 35 or the use of embodiment 35, wherein the immune effector cell is a monocyte selected from the group comprising THP-1 and U937, preferably, a differentiated THP-1 and U937 cell.

39. The method of any of embodiments 1 or 4-38 or the use of any of embodiments 2-38, wherein in step b), the contacting is in either order or substantially at the same time, preferably, substantially at the same time.

40. The method of any of embodiments 1 or 4-39 or the use of any of embodiments 2-39, wherein in step b), the bead is first contacted with the immunoglobulin test composition to form a mixture, and the resulting mixture is then contacted with the immune effector cell.

41. The method of any of embodiments 1 or 4-39 or the use of any of embodiments 2-39, wherein in step b), the immune effector cell is first contacted with the immunoglobulin test composition to form a mixture, and the resulting mixture is then contacted with the bead.

42. The method of any of embodiments 1 or 4-39 or the use of any of embodiments 2-39, wherein in step b), the immune effector cell is first contacted with the bead to form a mixture, and the resulting mixture is then contacted with the immunoglobulin test composition.

43. The method of any of embodiments 1 or 4-42 or the use of any of embodiments 2-42, wherein the effector function is selected from the group consisting of cytokine production, phagocytosis of the beads, modulation of surface markers, NETose, ROS release and degranulation, optionally, production of at least one pro-inflammatory cytokine.

44. The method of any of embodiments 1 or 4-43 or the use of any of embodiments 2-43, wherein the effector function the effector function is cytokine secretion.

45. The method of any of embodiments 1 or 4-44 or the use of any of embodiments 2-44, wherein the cytokine is selected from the group comprising pro-inflammatory and anti-inflammatory chemokines and cytokines such as TNF-α, GM-CSF, RANTES, GRO-α, MIP1a, MIP113, MCP-1, MIF, M-CSF, ICAM-1, Serpin E-1, IL-1a, IL-1β, IL-1 ra, IL-2, IL-3, IL-4, IL-5, IL-6, IL-7, IL-8, IL-9, IL-10, IL-11, IL-12, IL-13, IL-17, IL-21, IL-22, IL-23, IFN-γ.

46. The method of embodiment 45 or the use of embodiment 45, wherein the cytokine is IL-8 and the immune effector cell is a neutrophil, e.g., a HL-60 cell.

47. The method of any of embodiments 1 or 4-46 or the use of any of embodiments 2-46, wherein the effector function is secretion of IL-8.

48. The method of embodiment 45 or the use of embodiment 45, wherein the cytokine is IL-6 and the immune effector cell is a macrophage.

49. The method of any of embodiments 1 or 4-43 or the use of any of embodiments 2-43, wherein the effector function is modulation of a surface marker selected from the group comprising CD14, CD11 b, CD35, CD86 and FcR, e.g., CD89, CD64, CD32 and CD16.

50. The method of any of embodiments 1 or 4-49 or the use of any of embodiments 2-49, wherein the effector function is compared with the effector function of a control test, carried out without contacting the bead coated with an antigen and an antibody specifically bound to said antigen with the immunoglobulin test composition, to determine a change in effector function.

51. The method of any of embodiments 1 or 4-50 or the use of any of embodiments 2-50, wherein the potency is immunomodulatory potency.

52. The method of any of embodiments 1 or 4-51 or the use of any of embodiments 2-51, wherein the inhibition of the effector function by the immunoglobulin test composition is positively correlated to the potency of the immunoglobulin test composition.

53. The method of any of embodiments 1 or 4-52 or the use of any of embodiments 2-52, wherein the inhibition of the effector function by the immunoglobulin test composition is positively correlated the efficiency of the immunoglobulin test composition in treatment of inflammation.

54. The method of embodiment 53 or the use of embodiment 53, wherein the inflammation is in the context of severe acute respiratory syndrome.

55. The method of embodiment 53 or the use of embodiment 53, wherein the inflammation is in the context of sepsis.

56. The method of any of embodiments 53-55 or the use of any of embodiments 53-55, wherein the inflammation is in the context of COVID-19.

57. The method of embodiment 53 or the use of embodiment 53, wherein the inflammation is in the context of sCAP caused by a bacterial and/or viral infection, e.g., a pneumococcal infection.

58. The method of any of embodiments 1 or 4-57 or the use of any of embodiments 2-57, wherein the potency of the immunoglobulin test composition is compared to the potency of a standard immunoglobulin composition, and the ratio of the potency of the immunoglobulin test composition to the potency of the standard immunoglobulin composition is the relative potency.

59. The method of embodiment 58 or the use of embodiment 58, wherein the standard immunoglobulin composition is a standard IgM and/or IgA containing immunoglobulin composition.

60. The method of any of embodiments 1-59 or the use of any of embodiments 2-59, wherein the potency is detected based on step c).

61. A method for preparing a standardized immunoglobulin composition comprising at least g/L immunoglobulins derived from a plurality of donors, comprising
i. pooling plasma or serum derived from a plurality of human donors to provide a pool;
ii. isolating and concentrating immunoglobulins from the pool to produce an immunoglobulin test composition;
iii. testing the potency of the immunoglobulin test composition of ii) by the method of any of embodiments 1 or 4-60, wherein said immunoglobulin test composition is discarded if the relative potency of said immunoglobulin test composition is not in a predetermined range; and
iv. optionally, adapting the potency of the immunoglobulin test composition to a desired potency; and/or packaging an amount of the immunoglobulin test composition, e.g., an amount having a desired potency.

62. A kit for carrying out the method of any of embodiments 1 or 4-60, comprising
the bead, the antigen and the antibody,
a standard immunoglobulin composition comprising at least 30 g/L immunoglobulins derived from a plurality of human donors, optionally, an IgM and/or IgA containing immunoglobulin composition.

63. A kit of embodiment 62, wherein the bead is coated with the antigen.

64. A kit of embodiment 62, wherein the bead is coated with an antigen and an antibody is specifically bound to said antigen.

65. A kit of embodiment 62, wherein the bead, the antigen and the antibody are present in separate containers.

66. A kit of any of embodiments 62-65, wherein the standard immunoglobulin composition comprises IgG, IgM and/or IgA, wherein, preferably, the immunoglobulin composition comprises about 50-60% (w/total antibody w) IgG, about 20-25% IgM and about 18-23% IgA, e.g., about 56% IgG, about 23% IgM and about 21% IgA.

67. A kit of any of embodiments 62-66, further comprising immune effector cells expressing FcR selected from the group comprising HL60 cells, THP-1 and U937, preferably, HL-60 cells.

68. A composition comprising a human immunoglobulin composition comprising at least 30 g/L immunoglobulins derived from a plurality of donors and a bead coated with an antigen and an antibody specifically bound to said antigen and, optionally, immune effector cells expressing FcR.

69. The composition of embodiment 68, wherein the human immunoglobulin composition comprises about 50-60% (w/total antibody w) IgG, about 20-25% IgM and about 18-23% IgA, e.g., about 23% IgM, about 56% IgG and about 21% IgA.

### DESCRIPTION OF THE FIGURES

FIG. 1 Schematic overview of the method of the invention. Fluorescent latex beads were coated with an antigen, e.g., SARS-CoV-2 spike protein to generate antigen-coated beads. Antibodies to the antigen, e.g., anti SARS-CoV-2 spike protein antibodies (source: convalescent plasma donation or recombinant antibody) were added to generate an immune complex, here, a SARS-CoV-2 like immune complex. These immune complex were phagocytosed FcγRII and FcαRI dependently by immune effector cells, e.g., HL60 cells. The phagocytosis leads to induction of effector functions, e.g., inflammatory cytokine release, by the immune effector cell. This can be reduced by addition of an immunoglobulin test composition such as an IVIG or IVIG-AM preparation. Immunoglobulins added may inhibit immune complex uptake, target inhibitory cell signalling, modulate cell phenotype or gene expression, and neutralize cytokines. Immunomodulation may be determined by monitoring, e.g., reduced pro-inflammatory cytokine release and bead uptake.

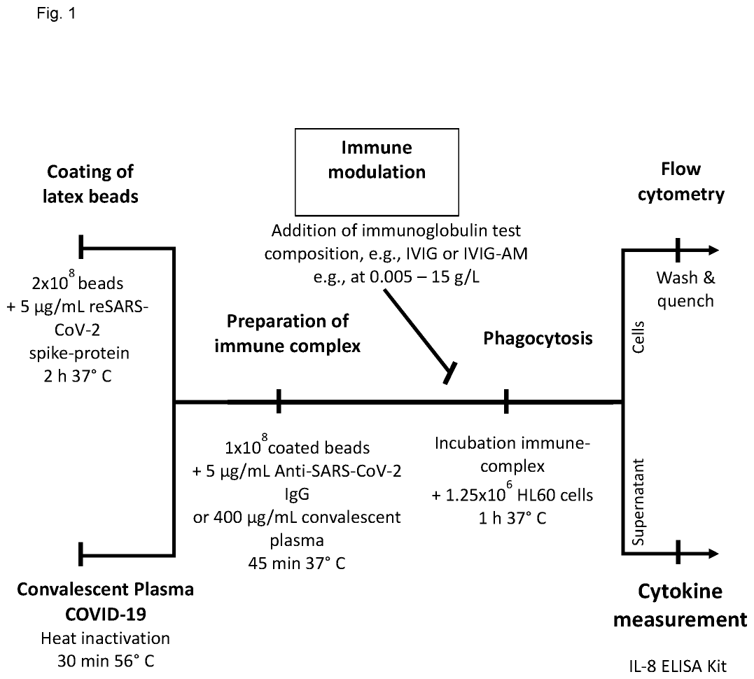

FIG. 2 Modulation of phagocytosis and cytokine secretion by an immunoglobulin test composition. Uptake of latex beads and 1×10⁸ fluorescent latex beads coated with 5 μg/mL reSARS-CoV-2 spike protein and bound by 5 μg/mL anti-SARS-CoV-2 chimeric IgG (A) or 400 μg/mL convalescent plasma from a COVID-19 patient to form a SARS-CoV-2-like immune complex coated on beads (B). Said beads with the SARS-CoV-2-like immune complex were added to neutrophil-like HL60 cells and phagocytosis was performed for 1 h. Uptake of beads was monitored by phagocytic index (mean fluorescent intensity multiplied with % of positive cells). Cellular inflammation was measured by IL-8 secretion in cell culture supernatant. Effects of IVIG (pointed bars and triangles) with IVIG-AM (striped bars and balls, lower line) were compared. Statistics: Two way ANOVA; Bonferroni's multiple comparisons test, p 0,001***, p 0,0001****, 95% confidence interval, n=2.

FIG. 3 Immunomodulation in COVID-19-like model by IVIG and IVIG-AM preparation. HL60 cells were incubated for 1 h with SARS-CoV-2 spike protein coated beads opsonized with COVID-19 plasma. IVIG (IgG Next Generation, Biotest AG) or IVIG-AM (trimodulin, Biotest AG) were added in the indicated concentrations to the cell immune-complex mixture. Cytokine release into cell culture supernatant was measured with IVIG-AM (black balls) or IVIG (white triangles) addition. Interleukin (IL)-8 (A), IL-10 (B), monocyte chemoattractant protein-1 (MCP-1) (C) and IL-1 receptor antagonist (IL-1ra) (D) were measured. Values represent mean of 6 independent experiments. Statistics: Two way ANOVA; Tukeys multiple comparisons test, Significance was quantified as p-values with asterisks: * p≤0.05, ** p≤0.01, *** p≤0.001, **** p≤0.0001; with 95% confidence interval, ns=not significant.

### EXAMPLES

### Example 1: Manufacture of an IgA and kW Containing Immunoglobulin Composition (IVIG-AM)

Human blood plasma for fractionation (2000 l) from more than 500 donors was used as starting material. The plasma was transferred to the pooling area and pooled.

A cryoprecipitation step was performed in order to separate coagulation factors such as Factor VIII, von Willebrand Factor, and Fibrinogen. In order to obtain the cryoprecipitate, the temperature of the plasma was adjusted under gentle stirring so that the temperature range was kept at 2±2° C. Under these conditions the cryoprecipitate remains undissolved in the thawed plasma. The cryoprecipitate was separated from the plasma by a continuously operating centrifuge such as a Westfalia separator.

From the supernatant of the cryoprecipitation step the Cohn fraction I/II/III was precipitated by ethanol precipitation as follows:

The temperature of the centrifugation supernatant remaining after separation of the cryoprecipitate was adjusted to 2±2° C. The protein solution was adjusted to pH 5.9. Subsequently, the temperature was lowered to −5° C. and ethanol was added to a final concentration of 20% by volume. Under constant slow stirring in a stainless steel vessel, Cohn Fraction I/II/III was precipitated. The Cohn Fraction I/II/III precipitate was separated from the supernatant by filtration with depth filter sheets under addition of filter aid such as Perlite or Diatomaceous Earth, using a filter press. The Cohn fraction I/II/III was recovered from the filter sheets. This Cohn fraction I/II/III precipitate comprised all immunoglobulins (IgG, IgA, IgM) in approximately the following percentages: 75% IgG, 13% IgM and 12% IgA.

90 kg of the obtained Cohn fraction I/II/III precipitate were resuspended in 450 kg of 0.1 M sodium acetate puffer pH 4.8 and mixed for 60 minutes at 22° C. The pH of the suspension was adjusted to 4.8 with acetic acid.

In the following a treatment with octanoic acid was performed. The solution was treated by addition of 7.7 kg octanoic acid at room temperature. The octanoic acid was added slowly and the protein solution was further mixed for 60 minutes, using a vibrating mixer (Vibromixer®, Size 4, Graber+Pfenniger GmbH, Vibromixer adjusted to level 2-3).

A calcium phosphate treatment was performed in order to complete the octanoic acid reaction as follows:

Approximately 1.1 kg Ca 3 (PO 4) 2 were added and the protein solution was further mixed for more than 15 minutes and filtered over depth filter sheets. The filtrate was further processed. The obtained protein solution was subjected to ultrafiltration to a protein concentration of about 50 g/I. The protein solution was diafiltered against 0.02 M sodium acetate buffer pH 4.5 and afterwards adjusted to a protein concentration of about 40 g/I.

The protein solution was treated at pH 4.0 in order to inactivate viruses as follows: The pH was adjusted to pH 4.0 using 0.2 M HCl, and the resulting solution was incubated for 8 hours at 37° C. The resulting protein solution contains immunoglobulins with the following distribution: 90% IgG, 5% IgA, and 5% IgM.

The obtained protein solution was further processed by anionic exchange chromatography using a macroporous anion exchange resin in order to remove accompanying proteins and to obtain an IgG- and IgM-enriched immunoglobulin compositions. Per kilogram of the intermediate protein solution 0.00121 kg of tris(hydroxymethyl)aminomethane (Tris) were added and dissolved while stirring and the conductivity was adjusted to 6 mS/cm with solid NaCl. The protein solution was adjusted to pH 7.1 by adding 1 M NaOH. A macroporous anion exchange resin (POROS® 50 HQ anion exchange resin, Life Technologies, bed height of the column: 25 cm) was equilibrated with a 10 mM Tris buffer solution (pH 7.1, 50 mM NaCl, at a linear flow rate of 800 cm/h). The protein solution was loaded on the anion exchange resin with 40 g protein per liter of resin. The column was washed with the equilibration buffer (10 mM Tris, 50 mM NaCl, pH 7.1, at 800 cm/h).

An IgG-enriched immunoglobulin composition was obtained in the flow-through fraction and was further processed as described in Example 2 below.

An IgM-enriched fraction was eluted by increasing the conductivity as follows: 10 mM Tris buffer solution with 300 mM NaCl at pH 7.1 is used at 800 cm/h to elute the IgM-enriched fraction. The eluted fraction contained 58% IgG, 22% IgA and 20% IgM.

The protein solution was filtered through a Pall, Ultipor VF DV50 filter as a virus removal step. The filtrate was further processed by UVC light treatment at 254 nm, using a flowthrough UVivatech process device (Bayer Technology Services/Sartorius) at a UVC dose of 225 J/m² for further virus inactivation. The flow velocity through the UVC reactor was calculated using the manufacturer's instructions. The irradiated protein solution was concentrated to a protein concentration of 50 g/I by ultrafiltration (and was subjected to diafiltration (using 0.3 M glycine buffer pH 4.5). The final product was filtered through a 0.2 μm filter and was stored at 2 to 8° C.

The obtained immunoglobulin composition had an IgM content of 22% by weight, an IgA content of 22% by weight and an IgG content of 56% by weight, based on the total immunoglobulin content, at an immunoglobulin concentration of 50 mg/ml. The ACA was 0.34 CH50/mg.

### Example 2: Manufacture of a Purified IgG Containing Immunoglobulin Composition (IVIG)

The IgG-enriched immunoglobulin composition collected as the flow through fraction of the macroporous anion exchange chromatography (POROS® 50 HQ) in Example 1 was adjusted to pH 5.5 and to a conductivity of 22-26 mS/cm with sodium acetate buffer and NaCl and then was further purified by cation exchange chromatography in a flow-through mode on a cation exchange resin (POROS® 50 HS). The binding capacity of this resin is defined as 100-3000 g/I, and chromatography was carried out at a load of 3000 g/I and a flow-rate of 800 cm/h.

The cation exchange column was equilibrated with acetate buffer solution (pH 5.5, adjusted to 22-26 mS/cm with NaCl). The protein solution was loaded to the column and washed with acetate buffer (pH 5.5, adjusted to 22-26 mS/cm with NaCl). The flow through fraction and the wash are collected and further processed. The residual protein is eluted with 1.5 M NaCl.

The resulting protein solution was further processed by a nanofiltration step, in order to remove potentially present virus. A Planova BioEx 20 nm filter (Asahi Kasei) was used as a virus filter. More than 50 kg of the protein solution were filtered over a 0.1 m 2 filter area at a protein concentration of 10 g/I. The maximum pressure was set according to the manufacturer's instructions.

The resulting protein solution was subjected to a concentration step to >100 g/L by ultrafiltration and diafiltered into formulation buffer (0.3 M Glycine pH 5.0). The resulting protein solution was filtered through a 0.2 μm filter in order to control sterility.

Obtained immunoglobulin compositions were analysed for their potency using the method of the invention, i.e., they were analysed as immunoglobulin test compositions in the method of the invention.

### Example 3: Potency Assay

In brief, fluorescent latex beads were coated with an antigen, e.g., SARS-CoV-2 spike protein to generate antigen-coated beads. Antibodies to the antigen, e.g., anti SARS-CoV-2 spike protein antibodies (source: convalescent plasma donation or recombinant antibody) were added to generate a bead coated with an immune complex, here, a SARS-CoV-2 like immune complex. The beads coated with the immune complex were contacted with immune effector cells expressing at least one Fc, e.g., HL60 cells, in the presence or absence of an immunoglobulin test composition. The contact of the coated beads and resulting immune complex to the effector cells leads to induction of effector functions, e.g., inflammatory cytokine release or phagocytosis. For example, the coated beads incubated with specific antibodies to generate an immune complex were phagocytosed FcγRII and FcαRI dependently by the immune effector cells. The contact leads to induction of effector functions, e.g., inflammatory cytokine release, by the immune effector cell. This was reduced by addition of an immunoglobulin test composition such as an IVIG or IVIG-AM preparation, depending on the potency of said test composition. Immunomodulation caused by the immunoglobulins may be determined by monitoring, e.g., reduced pro-inflammatory cytokine release and bead uptake, preferably, IL-8 secretion.

Without intending to be bound by the theory, immunoglobulins added may e.g. inhibit immune complex uptake, target inhibitory cell signalling, modulate cell phenotype and neutralize cytokines.

Material and Methods

Differentiation of HL60 Cells

HL60 cells were cultivated in IMDM-Medium supplemented with 20% fetal bovine serum and 1% Penicillin (10.000 U/mL)/Streptomycin (10.000 μg/mL). After seeding, the cells were cultivated for 3-4 days at 37° C. in the incubator (5% CO₂). For subculturing, the cells were count and seeded with a density of 2×10 5 cells/mL in fresh medium and a new flask. Up to 50 mL cell suspension was cultivated in a T-75 flask, up to 70 mL in a T-175 flask.

The assays were started, e.g., after passage 4-5 with a viability 95%. Cultivation may be maintained, e.g., until cells reach passage 25.

Differentiation of HL60 cells was induced by resuspending a cell pellet in complete culture medium supplemented with 1.3% (v/v) cell culture grade DMSO to reach a cell density of 6×10⁵ cells/mL. These cells were incubated for 3-5 days at 37° C. in the incubator to reach a neutrophil-like phenotype. Differentiation was monitored by flow cytometry.

Optionally, the HL-60 cells cultured in DMSO as described herein were further matured stimulated with LPS, e.g., 500 ng/mL LPS. The stimulation was for 6-48 h, preferably, at least over 24-48 hours.

Isolation of Primary Human Neutrophils

For the isolation of primary human neutrophils, fresh whole blood donations were used. The isolation was performed by using the MACSxpress® Whole Blood Neutrophil Isolation Kit (Miltenyi-Biotec, Bergisch Gladbach, DE). Isolation was performed according to manufacturer's instruction. In short: 8 mL of full blood donation was mixed with 4 mL of the Isolation Mix-cocktail and incubated for 5 min on a tube rotator, then the tube was transferred in the magnetic field of MACSxpress® Separator for 15 min. There the magnetic labeled cells were separated from the neutrophils and the red blood cells sediment to the bottom of the tube.

The untouched neutrophils were pipetted in to a clean tube and centrifuged for 5 min at 350×g. Red blood cells were lysed by resuspending the cell pellet in 10 mL red-blood-cells lysis solution for 10 min. The reaction was stopped by addition of 25 mL D-PBS. After centrifugation the cells were resuspended in RPMI-1640 medium and the cell number were determined. The purity of the cells and the success of the isolation was controlled by flow cytometry. The isolated neutrophils were cultured in RPMI-1640 supplemented with 10% FBS and 1% Penicillin (10.000 U/mL)/Streptomycin (10.000 μg/mL) or were directly used in cell-based assays.

The assay of the invention can be carried out with primary neutrophils.

Preparation of Antigen-Coated Beads and Immune Complexes Thereof

For the preparation of antigen-coated beads fluorescent latex beads were coated with recombinant antigen, e.g., virus protein such as spike protein from SARS-CoV-2. Coating of carboxylate-modified polystyrene yellow fluorescent latex beads (L4655-1ML Sigma-Aldrich) was done by same day as experiments. First beads were washed twice with a buffer comprising 50 mM 2-(N-Morpholino)ethanesulfonic acid (MES) and 1.3 mM N-Ethyl-N′-(3-dimethylaminopropyl) carbodiimide hydrochloride (EDAC) in Aqua dest. pH 6.1. MES/EDAC-Buffer has to be prepared freshly because of the low stability of EDAC.

The bead suspension was transferred into a 15 mL falcon tube and centrifuged (4.700×g for 20 min). Supernatant was discarded and beads were resuspend in MES/EDAC buffer. This wash step was performed twice. To completely dissolve the beads, a short incubation in ultrasonic cleaner was done for 1:30 min. Coating of latex beads was performed by adding 5 μg/mL antigen, e.g., reSARS-CoV-2 full-length spike protein (SPN-052H4, Acro Biosystems) to 2×10⁸ beads/mL in 1 mL MES/EDAC-Buffer. The mixture was incubated for 2 h at 37° C. with 500 rpm in a heat block in the dark.

To block remaining binding sites, the double volume of 2% (w/v) BSA/PBS was added to the bead/spike-protein mixture after incubation. After a centrifugation at 4,700×g, 15 min, the pellet was resuspended in 2% (w/v) BSA/PBS and centrifuged again. Finally the pellet was resuspended in 0.1% (w/v) BSA/PBS to 1×10⁸ beads/mL.

Heat inactivation of plasma was done by incubation for 30 min at 56° C., followed centrifugation at 4,000×g for 10 min. Precipitate was discarded and supernatant used for preparation of the immune complexes.

Preparation of the immune complex with SARS-CoV-2 virus-like particles was done by adding either 5 μg/mL specific anti-SARS-CoV-2 chimeric monoclonal antibody (40150-D001-50, SinoBiological) or 400 μg/mL heat inactivated convalescent COVID-19 plasma (Deutsches Rotes Kreuz) to the coated and blocked beads. The beads were incubated for 45 min at 37° C. with the antibody or the plasma.

As controls, antibodies (negative for SARS-CoV-2) or BSA were mixed with blocked beads and incubated as described above.

The binding of antibodies to the antigen-coated beads is essential. IgG, IgA and IgM could be detected on the surface of beads prepared as described herein by flow cytometry (data not shown).

Potency Assay

HL60 cells were prepared as described above.

1*10⁸ latex beads were coated with SARS-CoV-2 spike protein, as described above, and immune complexes formed by incubation with 5 μg/mL anti-SARS CoV-2 IgG or, preferably, 400 μg/mL convalescent heat-inactivated plasma, e.g., for 45 min at 37° C. The antigen-coated beads bound by antibodies (beads coated with immune complexes) were washed with D-PBS. After resuspension in 100 μL IMDM without FBS, the beads were added to the HL-60 cells and incubated. Directly after addition of the cells, an immunoglobulin test composition, e.g., IVIG or IVIG-AM at 0.005-15 g/L (or a buffer control) was added. Remaining volume (to the highest concentration) was filled up with formulation buffer.

After incubation, e.g., for 1 h at 37° C., the supernatant was analysed for cytokine secretion, and the cells were analysed by flow cytometry.

Measurement of Cytokine Release

For measurement of the cytokine-release, cell culture supernatant from the co-incubation of the beads coated with the immune complex and the immune effector cells, e.g., the HL-60 cells, may be analysed by different techniques, e.g., cytokine arrays or multiplex assays may be performed. Quantification of selected cytokines was done with commercial human cytokine ELISA-Kits.

For example, quantification of IL-8 levels in cell culture supernatants was performed with an Abcam human IL-8 ELISA Kit, based on a classical sandwich ELISA. The assay was performed according to manufacturer's instructions. If needed, the supernatant was diluted before the analysis.

Results

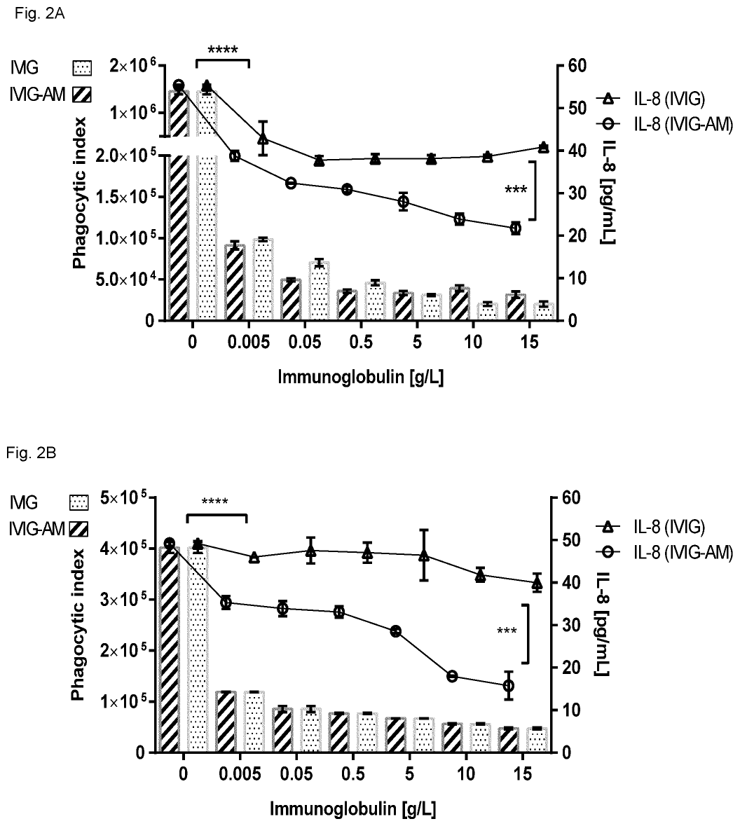

The uptake of fluorescent beads and IL-8 secretion by HL-60 cells incubated with different amounts of immunoglobulin test compositions, IVIG and IVIG-AM is shown in FIG. 2. The assay shows a significant and dose dependent decrease in phagocytic index, which means that the addition of classical IVIG and IVIG-AM lead to decreased uptake of beads. No clear difference in bead uptake between IVIG and IVIG-AM is detectable.

The release of IL-8 into cell culture supernatant was decreased by addition of both immunoglobulin test preparations. IVIG-AM reduces IL-8 level significant more than classical IVIG preparation. Therefore, IVIG-AM demonstrates stronger immunomodulatory properties then IVIG preparation in this assay when IL-8 secretion is analysed.

By replacing chimeric anti-SARS-CoV-2 IgG (FIG. 2A) against convalescent plasma (FIG. 2B), the systems was expanded to bind immunoglobulins IgG, IgA and IgM against SARS-CoV-2 spike protein on the beads. As described above, bead uptake and IL-8 level were monitored.

FIG. 2B demonstrates the particularly high immunomodulatory properties of IVIG-AM. Similar to the chimeric IgG model, the uptake of IgG, IgA and IgM bound latex beads was significant reduced by both IVIG and IVIG-AM. The modulation of IL-8 release by IVIG-AM is much stronger than the modulation by IVIG. The modulation of IL-8 release by IVIG-AM in this experiment, with heat-inactivated reconvalescent plasma was also stronger than shown with the chimeric anti-SARS-CoV-2 IgG model.

It was shown, via FcR Blocking experiments, that the convalescent plasma immune complex uptake and IL-8 secretion is FcαRI dependent. Thus, IgA species in convalescent plasma induce inflammation and this in turn could be modulated by the IgA component of immunoglobulin test compositions. Therefore the assay of the present invention shows an increased immunomodulatory potency of IVIG-AM compared to IVIG, which is in line with possible benefits for immunomodulatory treatment of COVID-19 patients with IVIG-AM over standard IVIG.

In summary, the data demonstrate the establishment of a new assay capable of determining the immunomodulatory potency of immunoglobulin preparations in a system modelling immune stimulation by a virus such as SARS-CoV-2. The analysis of cytokine secretion is believed to more closely mirror the physiologic relevance, as it is able to demonstrate the improved immunomodulatory potency of IVIG-AM. An improved immunomodulatory potency of IVIG-AM has also been shown in vivo, e.g., IgM-concentrate was developed and effectively tested for severe community acquired pneumonia (sCAP) (Welte et al., 2018), which has symptoms similar to COVID-19. Therefore IVIG, in particular, IVIG-AM could be an ideal treatment for severe COVID-19 patients. First clinical studies already show promising results in treatment of COVID-19 patients with IVIG preparations (Cao et al. 2020. Open Forum Infect. Dis. 7.; Xie et al. 2020. J. Infect. S0163-4453(20)30172-9).

### 1.1 Example 4: Addition of IVIG and IVIG-AM Preparation Reduces Inflammation

More aspects of immunomodulation in the method of the invention, as described herein, were investigated by the addition of various concentrations of immunoglobulin test compositions. IgG containing IVIG (IgG Next Generation, Biotest AG), as well as IgG, IgA and IgM containing IVIG-AM (trimodulin, Biotest AG) were compared. Used lots were tested negative for anti-SARS-CoV-2 neutralizing antibodies. IL-8, IL-10, MCP-1, and IL-1ra release were measured as markers of inflammation.

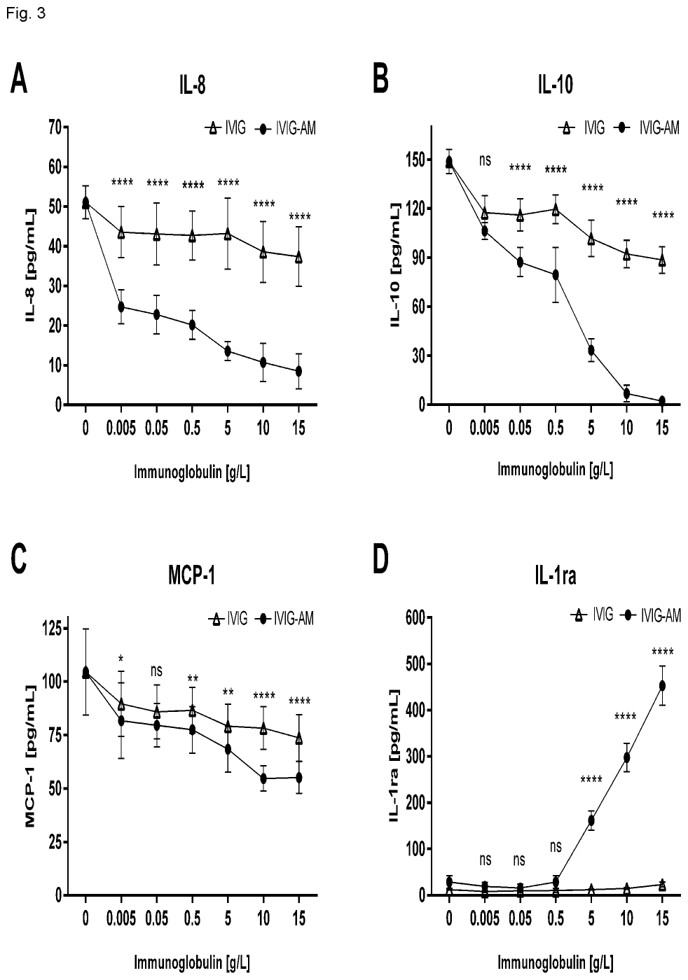

Addition of IVIG or IVIG-AM to HL60 cells significantly and equally decreased bead uptake of SARS-CoV-2-like particles opsonized with COVID-19 plasma (data of this experiment not shown). The corresponding cytokine release is also affected: IL-10, MCP-1 and IL-8 level are reduced by IVIG and significantly more by IVIG-AM addition (FIG. 3A-C). IL-1ra is strongly induced by IVIG-AM, but not by IVIG addition (FIG. 3D). The observed effects are dose dependent. Similar to neutrophil-like HL60 cells, primary neutrophils show dose-dependent reduced phagocytosis and decreased IL-8 release by IVIG-AM and IVIG addition, however no difference between IVIG-AM and IVIG was observed (data not shown). Thus, depending on the aim of the assay, the skilled person can choose to use or not to use a certain type of effector cells.

Thus, HL60 cells are particularly advantageous effector cells, if potency of IgA- and/or IgM-containing immunoglobulin test compositions is to be analysed. In this case, also, the effector function determined can be the release of different pro- or anti-inflammatory cytokines such as IL8, IL-10, MCP-1 and/or IL-1ra. For example, as shown in FIG. 3D, if the immunoglobulin test composition essentially comprises only IgG (such as in a typical commercial IVIG preparation), it is preferred that an effector function other than IL-1ra release is determined.

1. A method for testing potency of an immunoglobulin test composition, the method comprising
a) providing a bead coated with an antigen and an antibody specifically bound to said antigen,
b) contacting said bead with said immunoglobulin test composition and with an immune effector cell expressing at least one Fc-Receptor (FcR), and
c) determining an effector function of the immune effector cell.

2. (canceled)

3. The method of claim 1, wherein the antigen is a viral surface protein.

4. The method of claim 1, wherein said antibody specifically bound to said antigen is an antibody from a patient who has been infected with a pathogen expressing said antigen, or recombinant antibody, preferably, heat-inactivated plasma of a convalescent COVID-19 patient.

5. The method of claim 1, wherein the bead is selected from the group consisting of latex beads, agarose beads, glass beads and gold beads, preferably, latex beads.

6. The method of claim 1, further comprising preparing a bead coated with the antigen by incubating a bead with the antigen, wherein, preferably, the antigen is covalently linked to the bead, wherein, optionally, free binding sites on the bead are blocked after said incubation.

7. The method of claim 1, further comprising preparing the bead coated with an antigen and an antibody specifically bound to said antigen by incubating the bead coated with the antigen with antibodies to said antigen.

8. The method of claim 1, wherein the immunoglobulin test composition is a polyclonal immunoglobulin composition, wherein, optionally, the immunoglobulin test composition is plasma, e.g., from a convalescent patient who had a disease associated with the antigen.

9. The method of claim 1, wherein the immunoglobulin test composition comprises at least 30 g/L immunoglobulins and is derived from a plurality of human donors,
and, optionally, comprises IgG, IgM and/or IgA, preferably, all three classes, e.g., the percentage of IgM and/or IgA being about 5-90% (w/total antibody w), respectively.

10. The method of claim 1, wherein the immunoglobulin test composition is derived from plasma or serum, optionally, plasma.

11. The method of claim 1, wherein the effector function is compared with the effector function of a control test carried out without contacting the bead coated with an antigen and an antibody specifically bound to said antigen with the immunoglobulin test composition to determine a change in effector function.

12. The method of claim 1, wherein the immune effector cell is selected from the group consisting of neutrophils, eosinophils, monocytes, macrophages, and dendritic cells, preferably, HL60 cells.

13. The method of claim 1, wherein the effector function is selected from the group consisting of cytokine production, phagocytosis of the beads, modulation of a surface marker, NETose, ROS release and degranulation, wherein, preferably cytokine secretion is determined.

14. The method of claim 1, wherein the effector function is production of IL-8, preferably, secretion of IL-8.

15. The method of claim 1, wherein the potency is immunomodulatory potency.

16. The method of claim 1, wherein the inhibition of the effector function by the immunoglobulin test composition is positively correlated to the potency of the immunoglobulin test composition, and, optionally, to efficiency of the immunoglobulin test composition in treatment of inflammation, optionally, in the context of COVID-19.

17. The method of claim 1, wherein the potency of the immunoglobulin test composition is compared to the potency of a standard immunoglobulin composition, and the ratio of the potency of the immunoglobulin test composition to the potency of the standard immunoglobulin composition is the relative potency,
wherein the standard immunoglobulin composition preferably is a standard IgM and/or IgA containing immunoglobulin composition.

18. A method for preparing a standardized immunoglobulin composition comprising at least 30 g/L immunoglobulins derived from a plurality of donors, comprising
i. pooling plasma or serum derived from a plurality of human donors to provide a pool;
ii. isolating and concentrating immunoglobulins from the pool to produce an immunoglobulin test composition;
iii. testing the potency of the immunoglobulin test composition of ii) by the method of claim 17, wherein said immunoglobulin test composition is discarded if the relative potency of said immunoglobulin test composition is not in a predetermined range; and
iv. optionally, adapting the potency of the immunoglobulin test composition to a desired potency; and/or packaging an amount of the immunoglobulin test composition, e.g., an amount having a desired potency.

19. A kit for carrying out the method of claim 1, comprising
the bead, the antigen and the antibody, wherein, optionally, the bead is coated with the antigen, or the bead is coated with an antigen and an antibody is specifically bound to said antigen,
a standard immunoglobulin composition comprising at least 30 g/L immunoglobulins derived from a plurality of human donors, optionally, an IgM and/or IgA containing immunoglobulin composition,
and, optionally, immune effector cells expressing FcR selected from the group comprising HL60 cells.

20. The method of claim 3, wherein the antigen is a SARS-CoV-2 surface protein.

21. The method of claim 20, wherein the antigen is a SARS-CoV-2 spike protein.
